(19) United States
(12) Patent Application Publication
(10) Pub. No.: US 2020/0191803 A1
(43) Pub. Date: Jun. 18, 2020

(54) MARKERS FOR DISEASE AND DISEASE EXTENT IN INFLAMMATORY BOWEL DISEASE

(71) Applicant: UNIVERSITY OF OTTAWA, Ottawa (CA)

(72) Inventors: Joseph Michel Daniel FIGEYS, Ottawa (CA); Alain Christophe STINTZI, Ottawa (CA); David R. MACK, Ottawa (CA); Amanda Elizabeth Starr, Ottawa (CA); Shelley A. Deeke, Ottawa (CA)

(21) Appl. No.: 16/622,051

(22) PCT Filed: Jun. 14, 2018
(86) PCT No.: PCT/CA2018/050716

§ 371 Date: Dec. 12, 2019

Related U.S. Application Data
(60) Provisional application No. 62/520,652, filed on Jun. 16, 2017.

(51) Int. Cl. G01N 33/68

(52) U.S. Cl. CPC G01N 33/6893; G01N 2800/065; G01N 2333/948; G01N 2800/52; G01N 2333/9104; G01N 2800/067; G01N 2333/908

(57) ABSTRACT

A method for determining a presence of inflammatory bowel disease in a subject. The method involves providing a gut sample obtained from a subject; measuring a level in said gut sample of one or more proteins, wherein said one or more proteins comprises at least one of: leukotriene A-4 hydrolase, catalase, transketolase, thioredoxin domain containing protein 17, vasodilator-stimulated phosphoprotein and thymosin beta-10; and comparing said measured level of each of said one or more proteins to a corresponding protein level for a normal subject. A method for determining a presence of pancolitis in a subject with ulcerative colitis is also provided.

The present application claims priority of U.S. provisional patent application No. 62/520,652 filed on Jun. 16, 2017.

### TECHNICAL FIELD

The present application relates to protein markers for inflammatory bowel disease (IBD), including namely ulcerative colitis (UC) and Crohn's disease (CD).

### BACKGROUND

Inflammatory Bowel Disease (IBD) encompasses two principal conditions: ulcerative colitis (UC) and Crohn's disease (CD). Some patients have features of both subtypes and are classified as IBD-unclassified (IBD-U) (Gastroenterology, 2007. 133(5): p. 1670-89). UC is defined by continuous mucosal inflammation starting in the rectum and restricted to the colon while CD inflammation can occur anywhere in the gastrointestinal tract, involves full thickness of the bowel wall and often with skip lesions (Gastroenterol Clin North Am, 2009. 38(4): p. 611-28; Gastroenterology, 2007. 133(5): p. 1670-89).

One of the primary tools used for both diagnosis and IBD management is endoscopy (World J Gastrointest Endosc, 2012. 4(6): p. 201-11). Endoscopy enables both visualization of the mucosa and access for mucosal biopsies to diagnose disease, to define disease extent and activity, and to monitor disease progression. The diagnostic accuracy from colonoscopy ranges from 60 to 74% (J Clin Pathol, 2002. 55: p. 955-60). Other diagnostic approaches include radiological imaging and histological examination of mucosal biopsies in the differentiation of IBD subtypes (e.g non-caseating submucosal granuloma). However, 10% of patients (Registry. Dtsch Arztebl Int 2015; 112:121-7) have ambiguous diagnosis using these approaches and are instead classified as IBD-unclassified (IBD-U) patients (J Pediatr Gastroenterol Nutr 2014; 58:795-806). Accurate and early diagnosis is essential for proper disease management. The goal of IBD treatment is to bring active disease into remission, prevent follow-up relapse (flare-ups) and prevent complications of disease. The choice of treatment depends on disease subtype (CD versus UC), disease location, severity of disease, disease complications and individual host factors (e.g. nutritional and growth status, pubertal status, child's age and size, medication allergies, co-morbid conditions) (J Pediatr Gastroenterol Nutr, 2010, S1-S13. The American Journal of Gastroenterology, 2011. 106 Suppl 1: p. S2-25; quiz S26. Gastroenterol Clin North Am, 2009. 38(4): p. 611-28). Current drug therapies consist of aminosalicylates, immune-modulators, corticosteroids, antibiotics and biological therapies (e.g. anti-TNFα monoclonal antibodies). One third of the cost associated with IBD is due to medical therapies (CCFC. 2008, report. p. 1-101) stressing the economic importance of an effective treatment and thereby an accurate diagnosis.

Genome wide association studies in both adults and pediatric patients have identified novel IBD-associated genes but only define 25% of the genetic risk for developing IBD and excepting for very young infants (i.e. <2 years of age), no unique genes have been discovered that define pediatric IBD from adult-onset IBD. IBD is a complex polygenic disease involving multiple risk gene loci (Nature genetics, 2008. 40(8): p. 955-62. Nature genetics, 2009. 41(12): p. 1335-40. Nature genetics, 2010. 42(4): p. 332-7). These loci encode genes involved in innate and adaptive immunity, autophagy, and maintenance of epithelial barrier integrity for those genes that have known function.

On average, children will suffer with IBD for at least four months before a diagnosis of IBD is established but oftentimes much longer, at which point the disease has often progressed to a more severe state (Dtsch Arztebl Int, 2015. 112(8): p. 121-7). The most frequently assayed biomarker used to distinguish IBD from non-inflammatory disorders or from extra-intestinal inflammatory disorders is fecal calprotectin, which outperforms blood markers (Erythrocyte Sedimentation Rate and C-reactive protein) in its ability to indicate intestinal inflammation³. While limited utility has been shown within the adult population, the diagnostic accuracy of calprotectin is inferior for pediatric patients⁴ where the specificity reaches only 0.682 in the context of suspected pediatric IBD⁵. The low specificity observed for IBD is due to similarly elevated levels of calprotectin measured in stool from children suffering from disorders including celiac disease, cystic fibrosis, infection, neoplasia and polyps⁶, allergic diseases^{7, 8}, and even in apparently healthy children⁹. Fecal calprotectin has also recently been implemented for the diagnosis of several allergic diseases in children, highlighting its lack of specificity in IBD diagnosis. In addition, the level of fecal calprotectin is influenced by age, with the highest levels observed in children under the age of four. Therefore, an elevated (positive) fecal calprotectin result necessitates further testing for suspected IBD cases, including endoscopy. Non-invasive biomarkers with the ability to lower the false positive rate associated with fecal calprotectin would be beneficial in order to reduce the number of unnecessary invasive colonoscopies, and thus avoid the risk, discomfort and economic burden associated with endoscopy.

Once an IBD diagnosis has been established, several other aspects of the disease must be assessed in order to select an appropriate therapeutic strategy, including disease severity and extent of disease (UC)^{10, 11}. To date, biomarkers able to determine extent of disease have not been implemented in the clinic. Instead, this aspect of diagnosis is achieved by endoscopy and imaging. UC is characterized by continuous mucosal inflammation limited to the colon, extending proximally from the rectum. The extent of disease in UC is defined as the macroscopic degree of inflammation in the colon, as assessed by colonoscopy; disease extent may partially dictate the method (oral or rectal) and type of treatment administered, and the recommended time to begin for monitoring for colorectal cancer (i.e. 8 to 10 years post-diagnosis of pancolitis).

In light of the above, there is a need for improved diagnostic methods for IBD, especially in children, to decrease the number of unnecessary invasive endoscopies performed for IBD diagnosis. There is also a need to assess the extent of disease in UC.

### SUMMARY

Applicant has discovered that proteins leukotriene A-4 hydrolase, catalase, transketolase and annexin A3 show increased expression in gut samples obtained from subjects with IBD when compared to corresponding protein expression levels in gut samples obtained from subjects without IBD. Therefore, applicant has discovered that these proteins may be used as reliable biomarkers to indicate if a patient has or does not have inflammatory bowel disease. These biomarkers may be used for disease diagnosis, treatment strategy and treatment responsiveness. Disease detection analysis by measuring the relative expression of these biomarkers may be performed in gut samples such as, for example, mucosal luminal interface samples, and stool samples.

Moreover, Applicant has discovered that proteins leukotriene A-4 hydrolase, thioredoxin domain containing protein 17, vasodilator-stimulated phosphoprotein, and thymosin beta-10 may be used as biomarkers to determine if patients with ulcerative colitis show a presence or an absence of pancolitis. These biomarkers may be used for disease diagnosis, determine the severity of the disease, determine the extent of disease, treatment strategy, treatment responsiveness, determining disease remission and determining disease relapse. Disease detection analysis by measuring the relative expression of these biomarkers may be performed in gut samples such as, for example, mucosal luminal interface samples, and stool samples.

Moreover, applicant has also discovered that thioredoxin domain containing protein 17, vasodilator-stimulated phosphoprotein, and thymosin beta-10 may also be used as biomarkers to detect IBD in a subject. These biomarkers may be measured in gut samples, such as mucosal luminal interface (MLI) samples and/or stool samples. One or more of these biomarkers (thioredoxin domain containing protein 17, vasodilator-stimulated phosphoprotein, and thymosin beta-10) may also be used in combination with one or more of leukotriene A-4 hydrolase, catalase, transketolase and annexin A3 to detect IBD, wherein the sensitivity and specificity may increase when more than one biomarker is used (up to 4 biomarkers may be used to reach optimal specificity and sensitivity).

A first broad aspect is a method for determining a presence of inflammatory bowel disease in a subject. The method involves providing a gut sample obtained from a subject. The method also involves measuring a level in the gut sample of one or more proteins, wherein the one or more proteins comprises at least one of: leukotriene A-4 hydrolase, catalase, transketolase, thioredoxin domain containing protein 17, vasodilator-stimulated phosphoprotein and thymosin beta-10. The method includes comparing the measured level to a predetermined level to provide an indication of presence of disease.

In some embodiments, the determining may be used to obtain an indication on remission of the disease.

In some embodiments, the determining may be used to obtain an indication of relapse of the disease.

In some embodiments, the one or more proteins may be leukotriene A-4 hydrolase and a measured level in the gut sample of leukotriene A-4 hydrolase higher than a predetermined protein level of leukotriene A-4 hydrolase corresponding to a healthy subject may be indicative of disease. The one or more proteins may be catalase and a measured level in the gut sample of catalase higher than a predetermined protein level of catalase corresponding to a healthy subject may be indicative of disease. The one or more proteins may be transketolase and wherein a measured level in the gut sample of transketolase higher than a predetermined protein level of transketolase corresponding to a healthy subject may be indicative of disease. The one or more proteins may be annexin A3 and wherein a measured level in the gut sample of annexin A3 higher than a predetermined protein level of annexin A3 corresponding to a subject patient may be indicative of disease.

In some embodiments, the measuring may be measuring each of leukotriene A-4 hydrolase, catalase, transketolase and annexin A3. In some embodiments, the measuring may be measuring one or more selected proteins, wherein said one or more selected proteins may be at least one of leukotriene A-4 hydrolase, catalase, transketolase and annexin A3.

In some embodiments, the measuring may be measuring a level in the gut sample of two or more proteins, wherein the two or more proteins may be at least two of: leukotriene A-4 hydrolase, catalase, transketolase, annexin A3, thioredoxin domain containing protein 17, vasodilator-stimulated phosphoprotein and thymosin beta-10. The measuring may be measuring a level in the gut sample of three or more proteins, wherein the three or more proteins may be at least three of: leukotriene A-4 hydrolase, catalase, transketolase, annexin A3, thioredoxin domain containing protein 17, vasodilator-stimulated phosphoprotein and thymosin beta-10. The measuring may be measuring a level in the gut sample of four proteins, or four or more proteins, wherein the four proteins are selected from: leukotriene A-4 hydrolase, catalase, transketolase, annexin A3, thioredoxin domain containing protein 17, vasodilator-stimulated phosphoprotein and thymosin beta-10. In some embodiments, the measuring may be measuring a level in the gut sample of two or more proteins, wherein the two or more proteins may be at least two of: leukotriene A-4 hydrolase, catalase, transketolase and annexin A3. The measuring may be measuring a level in the gut sample of three or more proteins, wherein the three or more proteins may be at least three of: leukotriene A-4 hydrolase, catalase, transketolase and annexin A3.

In some embodiments, the measuring may include using an immunoassay. The immunoassay may be ELISA. In some embodiments, the measuring may be using semi-quantitative immunoblotting. The measuring may include using mass spectrometry.

In some embodiments, the gut sample may be a mucosal luminal interface sample. The gut sample may be a stool sample.

The subject may be a pediatric subject. The subject may be an adult subject.

A second broad aspect is a method of treating inflammatory bowel disease in a subject involving determining whether the subject has inflammatory bowel disease according to the method for determining a presence of inflammatory bowel disease in a subject as described herein, and administrating to the patient a compound pharmaceutically effective against the inflammatory bowel disease. In some embodiments, the administering may involve administering a pharmaceutically effective amount of a compound selected from aminosalicylates, immunomodulators, anti-integrins, anti-cytokines, enteral feed programs, corticosteroids, antibiotics, monoclonal antibodies (e.g. anti-TNFα, anti-IL12/23, anti-integrin), or a combination thereof.

In some embodiments, where the subject was determined to have, at a time prior to obtaining the gut sample, inflammatory bowel disease, the measuring to provide an indication of presence of disease may be to further determine if the disease is in remission or if remission is maintained.

In some embodiments, the measuring may be to further determine if relapse of the disease has occurred.

A third broad aspect is a method for determining presence or an indication of pancolitis in a subject with ulcerative colitis. The method involves providing a gut sample obtained from a subject with ulcerative colitis. The method entails measuring in the gut sample one or more proteins, wherein the one or more proteins comprises at least one of: leukotriene A-4 hydrolase, thioredoxin domain containing protein 17, vasodilator-stimulated phosphoprotein, and thymosin beta-10. The method includes comparing the measured level to a predetermined protein level to provide an indication of the presence or absence of pancolitis.

In some embodiments, the one or more proteins may be leukotriene A-4 hydrolase and a measured level in the gut sample of leukotriene A-4 hydrolase higher than a predetermined protein level of leukotriene A-4 hydrolase corresponding to a subject without pancolitis may be indicative of pancolitis; the one or more proteins may be thioredoxin domain containing protein 17 and a measured level in the gut sample of thioredoxin domain containing protein 17 higher than a predetermined protein level of thioredoxin domain containing protein 17 corresponding to a subject without pancolitis may be indicative of pancolitis; the one or more proteins may be vasodilator-stimulated phosphoprotein and wherein a measured level in the gut sample of vasodilator-stimulated phosphoprotein higher than a predetermined protein level of vasodilator-stimulated phosphoprotein corresponding to a subject without pancolitis may be indicative of pancolitis; and/or the one or more proteins may be thymosin beta-10 and a measured level in the gut sample of thymosin beta-10 lower than a predetermined protein level of thymosin beta-10 corresponding to a subject without pancolitis may be indicative of pancolitis.

In some embodiments, the one or more proteins may be leukotriene A-4 hydrolase and wherein a measured level in the gut sample of leukotriene A-4 hydrolase higher than a predetermined protein level of leukotriene A-4 hydrolase corresponding to a subject without pancolitis may be indicative of pancolitis. The one or more proteins may be thioredoxin domain containing protein 17 and wherein a measured level in the gut sample of thioredoxin domain containing protein 17 higher than a predetermined protein level of thioredoxin domain containing protein 17 corresponding to a subject without pancolitis may be indicative of pancolitis. The one or more proteins may be vasodilator-stimulated phosphoprotein and a measured level in the gut sample of vasodilator-stimulated phosphoprotein higher than a predetermined protein level of vasodilator-stimulated phosphoprotein corresponding to a subject without pancolitis may be indicative of pancolitis. The one or more proteins may be thymosin beta-10 and a measured level in the gut sample of thymosin beta-10 lower than a predetermined protein level of thymosin beta-10 corresponding to a subject without pancolitis may be indicative of pancolitis.

In some embodiments, the measuring may involve measuring each of leukotriene A-4 hydrolase, thioredoxin domain containing protein 17, vasodilator-stimulated phosphoprotein, and thymosin beta-10. The measuring may involve measuring a level in the gut sample of two or more proteins, wherein the two or more proteins may be at least two of: leukotriene A-4 hydrolase, thioredoxin domain containing protein 17, vasodilator-stimulated phosphoprotein, and thymosin beta-10. The measuring may involve measuring a level in the gut sample of three or more proteins, wherein the three or more proteins may be at least three of: leukotriene A-4 hydrolase, thioredoxin domain containing protein 17, vasodilator-stimulated phosphoprotein, and thymosin beta-10.

In some embodiments, the gut sample may be a mucosal luminal interface sample. The gut sample may be a stool sample. The subject may be a pediatric subject. The subject may be an adult subject.

In some embodiments, the measuring may involve using an immunoassay. The immunoassay may be ELISA. The measuring may involve using semi-quantitative immunoblotting. The measuring may involve using mass spectrometry.

A fourth broad aspect is a method of treating ulcerative colitis in a subject involving determining whether the ulcerative colitis subject has pancolitis or does not have pancolitis according to the method for determining a presence or indication of pancolitis in a subject with ulcerative colitis as described herein, and administrating to the patient a compound pharmaceutically effective against: pancolitis; or ulcerative colitis without pancolitis, the administration tailored in accordance with the determined presence or absence of pancolitis. The administering may involve administering a pharmaceutically effective amount of a compound selected from aminosalicylates, immunomodulators, anti-cytokines, enteral feed programs, corticosteroids, anti-integrins, antibiotics, monoclonal antibodies (e.g. anti-TNFα, anti-IL12/23, anti-integrin), or a combination thereof.

A fifth broad aspect is a method for determining the efficacy of a treatment of inflammatory bowel disease in a patient suffering from the disease, the treatment comprising the administration of aminosalicylates, immunomodulators, anti-cytokines, enteral feed programs, corticosteroids, anti-integrins, antibiotics, monoclonal antibodies (e.g. anti-TNFα, anti-IL12/23, anti-integrin), or a combination thereof. The method involves measuring a level in an indicative gut sample, obtained from a patient, of one or more proteins, wherein the one or more proteins comprises at least one of: leukotriene A-4 hydrolase, catalase, transketolase, thioredoxin domain containing protein 17, vasodilator-stimulated phosphoprotein and thymosin beta-10. In some embodiments, two or more of the proteins, three or more of the proteins, or four or more of the proteins may be measured. In some embodiments, Annexin A3 may be measured. The method involves comparing the measured level to a corresponding protein level measured in a reference gut sample taken from the patient at a time prior to when the indicative gut sample was obtained; a predetermined protein level; a corresponding protein level associated with responders; and/or a corresponding protein level associated with non-responders. The method also involves assessing responsiveness to treatment as a function of the comparison.

In some embodiments, the comparing may involve further comparing the measured level with: corresponding protein level measured in a reference gut sample taken from the patient at a first time, the first time prior to the second time, (or with predetermined reference protein levels, such as that of controls) wherein the measured level in the indicative gut sample of each of the at least one of leukotriene A-4 hydrolase, catalase, transketolase, thioredoxin domain containing protein 17, vasodilator-stimulated phosphoprotein, annexin A3 lower than, and thymosin beta-10 higher than the corresponding protein level measured in the reference gut sample is indicative of responsiveness to treatment; corresponding protein levels of responders, wherein the measured level in the indicative gut sample of each of the at least one of leukotriene A-4 hydrolase, catalase, transketolase, thioredoxin domain containing protein 17, vasodilator-stimulated phosphoprotein, annexin A3 equal to or lower than, and thymosin beta-10 equal to or higher than the corresponding protein level the corresponding protein levels of responders is indicative of responsiveness to treatment; and/or corresponding protein levels of non-responders, wherein the measured level in the indicative gut sample of each of the at least one of leukotriene A-4 hydrolase, catalase, transketolase, thioredoxin domain containing protein 17, vasodilator-stimulated phosphoprotein, annexin A3 equal to or higher than and thymosin beta-10 equal to or lower than the corresponding protein levels of non-responders is indicative of non-responsiveness to treatment.

In some embodiments, the one or more proteins may be at least one of thioredoxin domain containing protein 17, vasodilator-stimulated phosphoprotein, thymosin beta-10 and leukotriene A-4 hydrolase. In some embodiments, the one or more proteins may be at least one of leukotriene A-4 hydrolase, catalase, transketolase and annexin A3.

In some embodiments, the corresponding protein levels of responders may be an average of responders' protein levels of the corresponding protein, and the corresponding protein levels of non-responders may be an average of non-responders' protein levels of the corresponding protein.

In some embodiments, the measuring involves performing an assay. In some embodiments, the patient may be a pediatric patient. In some embodiments, the patient may be an adult patient.

A sixth broad aspect is a method for determining the efficacy of a treatment of ulcerative colitis in a patient suffering from the disease, the treatment comprising the administration of aminosalicylates, immunomodulators, anti-cytokines, enteral feed programs, corticosteroids, anti-integrins, antibiotics, monoclonal antibodies (e.g. anti-TNF a, anti-IL12/23, anti-integrin), or a combination thereof. The method involves measuring a level, in a gut sample obtained from a patient, of one or more proteins, wherein the one or more proteins comprises at least one of: leukotriene A-4 hydrolase, thioredoxin domain containing protein 17, vasodilator-stimulated phosphoprotein, and thymosin beta-10. The method includes comparing the measured level in the gut sample to a predetermined protein level to indicate the presence or absence of pancolitis. The method includes assessing responsiveness of treatment with reference to a prior health condition of the patient wherein the assessment is indicative of responsiveness to treatment when the prior health condition was that the patient had pancolitis and the comparing the measured level in the gut sample indicates an absence of pancolitis; or the assessment is indicative of non-responsiveness to treatment when the prior health condition was that the patient did not have pancolitis and the comparing the measured level in the gut sample indicates a presence of pancolitis.

In some embodiments, the one or more proteins may be leukotriene A-4 hydrolase and a measured level in the gut sample of leukotriene A-4 hydrolase higher than a protein level of leukotriene A-4 hydrolase for a subject without pancolitis may be indicative of pancolitis; the one or more proteins may be thioredoxin domain containing protein 17 and a measured level in the gut sample of thioredoxin domain containing protein 17 higher than a protein level of thioredoxin domain containing protein 17 for a subject without pancolitis may be indicative of pancolitis; the one or more proteins may be vasodilator-stimulated phosphoprotein and a measured level in the gut sample of vasodilator-stimulated phosphoprotein higher than a protein level of vasodilator-stimulated phosphoprotein for a subject without pancolitis may be indicative of pancolitis; and/or the one or more proteins may be thymosin beta-10 and wherein a measured level in the gut sample of thymosin beta-10 lower than a protein level of thymosin beta-10 for a subject without pancolitis may be indicative of pancolitis.

In some embodiments, the measuring may involve performing an assay. In some embodiments, the patient may be a pediatric patient.

Another broad aspect is a method for determining a presence of inflammatory bowel disease in a subject. The method includes providing a gut sample obtained from a subject. The method includes measuring a level in the gut sample of two or more proteins, wherein the two or more proteins comprises at least two of: leukotriene A-4 hydrolase, Annexin A3, catalase, transketolase, thioredoxin domain containing protein 17, vasodilator-stimulated phosphoprotein and thymosin beta-10. The method includes comparing the measured level to a predetermined protein level to provide an indication of presence of disease.

In some embodiments, the providing an indication of presence of disease may further indicate if the disease is in relapse.

In some embodiments, the providing an indication of presence of disease may further indicate if the disease is in remission.

### BRIEF DESCRIPTION OF THE DRAWINGS

The invention will be better understood by way of the following detailed description of embodiments of the invention with reference to the appended drawings, in which:

FIG. 1 is a flow chart illustrating an exemplary set of steps to generate the IBD biomarker panel and an exemplary set of steps to obtain the biomarker panel for UC extent of disease.

FIG. 2 is a set of graphs showing age of patients included in cohort for the (A) ascending colon and (B) descending colon. No significant differences were observed between patient subgroups by one-way ANOVA.

FIG. 3 is a set of graphs of MS Data Evaluation. Median Log2 L/H normalized ratio (MLI proteins/super-SILAC reference proteome) ratio of proteins quantified in the (A) ascending colon and (B) descending colon. Dotted lines indicate 10-fold ratio threshold. Number of proteins quantified per patient in the (C) ascending colon and (D) descending colon. No significant differences in the number of proteins were observed between patient subgroups by one-way ANOVA.

FIG. 4 illustrates MS Data Evaluation. Namely, Pearson correlations of Q75 proteome log 2 (light/heavy) are shown in (A) for ascending colon and in (B) for descending colon of MLI samples. Hierarchical clustering of Pearson correlations are shown in (C) for ascending colon and in (D) for descending colon.

FIG. 5 is a set of graphs illustrating proteomic landscape alterations in treatment-naïve pediatric IBD: PCA of Q75 proteins from (A) ascending colon and (B) descending colon. CoN=without macroscopic inflammation, CoA=with macroscopic inflammation.

FIG. 6 is a set of graphs illustrating proteomic landscape evaluation at the colonic MLI. PCA of Q75 from (A) ascending colon and (B) descending colon. Intestinal MLI aspirate samples do not segregate according to gender in either colon sub-region.

FIG. 7 illustrates proteomic landscape evaluation at the colonic MLI. (A) number of features identified in ascending colon (left circle) and descending colon (right circle) between control patients and IBD patients with macroscopic evidence of inflammation. Top ten biological processes of discriminant features identified by comparison of control and IBD CoA patient samples by PLS-DA, enrichment is relative to Q75 from the (B) ascending colon and (C) descending colon.

FIG. 8 illustrates discriminant features identified by PLS-DA. (A) Number of features identified in the ascending colon (left circle) and in the descending colon (right circle) between control and IBD patient samples without macroscopic evidence of inflammation (CoN). Top biological processes in the (B) ascending colon and (C) descending colon of discriminant features identified by comparison of control and IBD CoN patient samples by PLS-DA; enrichment is relative to the Q75.

FIG. 9 is a protein interaction network (identified by the proteins' respective gene names) of features identified by PLS-DA of Control vs IBD CoA, common to both the current colonic mucosal-luminal interface (MLI) dataset and biopsy dataset. Grouping is based on relative IBD CoA/control expression levels between the two datasets. Border shading indicates relative expression in the biopsy data, whereas the internal shading represents the relative expression level from the MLI. High expression indicates elevated protein expression in IBD CoA compared to control, whereas low expression indicates decreased protein expression in IBD CoA compared to control. Squared boxes represent proteins involved in immune response. Small shape (22/26) indicates proteins that localize to the extracellular region. Arrows indicate protein-protein interaction.

FIG. 10 is a graph illustrating IBD Biomarker panel generation. The minimum number of proteins required to achieve maximum sensitivity and specificity in both colon sub-regions is indicated by the dotted line. Protein biomarker candidates were added based on highest combined (ascending colon and descending colon) AUC values, using the PLS-DA model to classify controls from IBD CoA.

FIG. 11 is a set of graphs illustrating an exemplary biomarker panel for suspected pediatric IBD diagnosis: (A) Relative expression levels of proteins included in IBD diagnosis biomarker panel. P values were generated by t-test ****p<0.0001. (B) Receiver operating characteristics curve utilizing panel of features listed in Table 3 for both the ascending colon and the descending colon. (C) Predictive class probabilities in each colon sub-region wherein samples predicted to be control are to the left of 0.5 and those predicted to be IBD are on the right of 0.5.

FIG. 12 is a set of graphs illustrating relative expression of proteins featured in the IBD diagnosis biomarker panel; (A) comparison with MLI samples lacking macroscopic evidence of inflammation (CoN) and (B) between CD (CoA) and UC (CoA). One-way ANOVA with Tukey's multiple comparison test. *p<0.05, **p<0.01, ***p<0.001, ****p<0.0001.

FIG. 13 is a set of graphs illustrating predictive class probabilities of Calprotectin (S100-A8 and S100-A9) using PLS-DA at the MLI in the (A) ascending colon and (B) descending colon.

FIG. 14 is a set of graphs illustrating an exemplary biomarker panel for extent of disease in UC (pancolitis vs non-pancolitis): (A) Relative expression levels of proteins included in the UC extent of disease biomarker panel. P values were generated by t-test ***p<0.001. (B) Receiver operating characteristics curve for differentiation of pancolitis from non-pancolitis utilizing the expression of proteins in UC extent of disease biomarker panel. (C) Predictive class probabilities of inflammatory status in the ascending colon (pancolitis vs non-pancolitis) wherein samples predicted to be inflamed are to the left of 0.5 and those predicted to be non-inflamed are on the right of 0.5.

FIG. 15 is a graph illustrating a minimum number of proteins required to achieve maximum sensitivity and specificity (indicated by the dotted line) for extent of disease biomarker panel (pancolitis vs non-pancolitis). Protein biomarker candidates added based on highest AUC values, using the PLS-DA model to classify UC CoN from UC CoA in the ascending colon.

FIG. 16 illustrates results with respect to the determination of select biomarkers in MLI samples and in stool: (A) Annexin A3 was validated by immunoblotting of descending colon MLI samples from an independent MLI cohort. (B) Stool samples obtained from a subset of IBD and control patients were analyzed by immunoblot (Annexin A3) and ELISA (LTA4H, Calprotectin), with quantitative data shown. Annexin A3 is shown relative to total protein, whereas ELISA results provide the absolute amount of protein. The dotted line indicates values above which Calprotectin is considered a positive result according to the manufacturer. The one patient that had commenced treatment prior stool collection is indicated by a (#).

FIG. 17 is a set of graphs illustrating the validation of select biomarkers in stool: (A) Catalase, (B) Leukotriene A-4 hydrolase and (C) transketolase, biomarker candidates proposed for the diagnosis of pediatric IBD, were validated by ELISA from a cohort consisting of independent patients and patients for which their MLI samples were utilized to develop the biomarker panel. P values were calculated using the Mann Whitney test. (D) The expression level of LTA4H in stool correlates with the PUCDAI. Analysis performed using Spearman two-tailed test.

FIG. 18 is a set of graphs illustrating the relative protein expression of biomarker candidates A) Annexin A3, B) Catalase, C) leukotriene A-4 hydrolase and D) transketolase in biopsy samples.

FIG. 19 is a graph of ROC curve for Control vs IBD CoA using a panel of proteins consisting of LTA4H, TXNDC17, TMSB10 and VASP, using AC MLI samples.

### DETAILED DESCRIPTION

The present application relates to novel biomarkers that have been identified to determine the presence of inflammatory bowel disease (IBD) in a human subject. Namely, the relative protein expression levels of leukotriene A-4 hydrolase, catalase, transketolase and annexin A3 were measured reliably in gut samples of subjects with IBD to be higher than in gut samples of normal control subjects. Therefore, these proteins were identified as biomarkers to determine the presence of IBD in a subject. By determining the presence of IBD, it is meant determining if a subject has IBD or does not have IBD (e.g. is healthy with respect to IBD).

The Applicant has also discovered that thioredoxin domain containing protein 17, vasodilator-stimulated phosphoprotein, and thymosin beta-10 may also be used as biomarkers to identify the presence of IBD in a subject.

Furthermore, it is shown that measuring one of these biomarkers alone may be sufficient to provide a reliable indication of IBD in a patient. However, using more than one biomarker may increase sensitivity and specificity of the test. When four biomarkers are used, the test may obtain a higher level of specificity and sensitivity in detection of IBD in a subject.

The applicant has also discovered that the relative protein expression levels of these IBD-detection biomarkers may be observed, in most cases, in both the ascending colon and in the descending colon. Moreover, given that mucosal luminal interface samples include proteins present in the colonic lumen, these biomarkers may also be found in stool as the stool transits through the lumen of the colon. Therefore, a less location-specific gut sample, such as a stool sample, may be used to measure protein expression levels of these biomarkers to detect IBD, instead and/or in addition to more localized samples, such as a mucosal luminal interface sample. For example, as described herein, similarly high relative expression levels of leukotriene A-4 hydrolase, catalase, annexin A3 and transketolase are measured in stool samples from subjects with IBD. Therefore, it is possible to utilize a stool sample, an example of a non-invasive sample, to measure the relative protein expression levels of these biomarkers to detect IBD. The higher relative protein expression level of transketolase may also be measured in a stool sample, such as by performing mass spectrometry when analyzing the stool sample. It will also be understood that the relative protein expression levels of thioredoxin domain containing protein 17, vasodilator-stimulated phosphoprotein, and thymosin beta-10 may also be measured in stool samples. As the relative expression levels of these biomarkers were measured in the mucosal luminal interface samples, these samples including proteins present in the colonic lumen, and given that stool transits with the lumen of the colon, the levels may also be measured in stool samples.

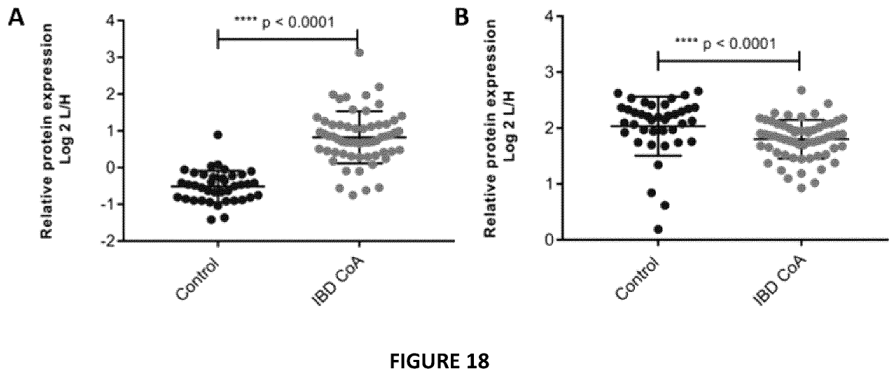

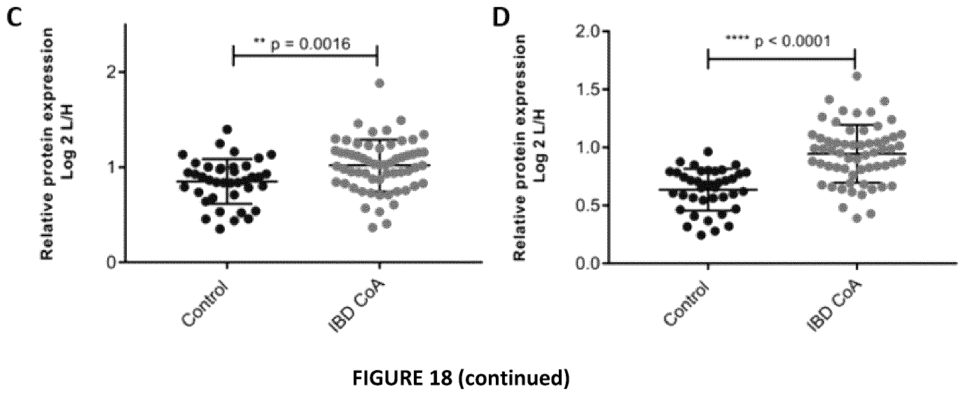

In some examples, the gut sample may be a biopsy sample, as shown by the relative protein expression levels of respectively leukotriene A-4 hydrolase, catalase, transketolase and annexin A3 shown in FIG. 18.

The expression level of the biomarkers as measured in a gut sample of a subject may be compared to a predetermined protein level to determine the presence of IBD. This predetermined protein level of the biomarkers may be a threshold or a reference. The threshold or reference may be related to protein expression level to distinguish between patients with disease and healthy. The threshold or reference may be related to protein expression levels in, for example, healthy subjects or controls, subjects with the disease, subjects in remission, etc. This comparison may provide an indication relative to the, e.g., presence, severity, remission, relapse of the disease. The predetermined protein level may also be, or related to, an absolute value of protein expression corresponding, for instance, to subjects with the disease, or healthy subjects.

Moreover, an increased relative protein expression level of the biomarkers may also be measured in patients without macro-inflammatory IBD and those with macro-inflammatory IBD. Therefore, these biomarkers may provide sufficient sensitivity to detect IBD in a patient without macro-inflammation.

In some examples, the biomarkers may also be used to establish a treatment for a patient having IBD, or to determine the patient's responsiveness to a given treatment for the disease. For instance, responsiveness may be established by taking one or more samples from the IBD-positive patient obtained at distinct times. A sample taken later may be compared to a sample obtained earlier from the same patient. A Protein expression level may be analyzed for the earlier sample, the later sample, and compared to a control sample for a normal patient. In some examples, only one sample may be needed, where the measured relative protein expression level may be compared to a predetermined threshold, such as that of a healthy patient (or that of responders) to determine responsiveness to treatment.

In some examples, a control sample is not needed, where the relative protein expression levels (or e.g. absolute protein concentrations of the biomarkers) of the biomarkers may be compared between the earlier sample and the later sample, or using the absolute protein concentration to compare with a predetermined level of the biomarkers, for instance, corresponding to healthy subjects (i.e. a reference). The difference in protein expression levels of the biomarkers between the earlier sample and the later sample may assist in telling if the patient is responding to the treatment. The results from the test to establish responsiveness may also be correlated with a disease severity index to establish if the disease is receding, maintaining the same severity or worsening.

In some examples, to determine responsiveness of treatment, the patient's sample may be compared with the biomarker expression levels taken from responders and/or non-responders. If the biomarker protein expression levels of the patient are similar to those of the responders, then this may serve as an indication of responsiveness to the treatment. However, if the biomarker protein expression levels are similar to those of the non-responders, then this may serve as an indication that the patient is not responding to the treatment. In some examples, an average or a median protein expression level for responders or non-responders may be used. The results from the test to establish responsiveness may also be correlated with a disease severity index to establish if the disease is receding, maintaining the same severity or worsening.

In other embodiments, comparing the relative protein expression levels of the biomarkers with a predetermined protein level may be used to assess if remission has been induced in the patient (e.g. such as in response to a given treatment), or if remission is maintained in the patient. For instance, the predetermined protein level may be that of one or more of the biomarkers as observed in remission cases (e.g. a predetermined level achieved by analyzing relative protein expression levels of the biomarkers in IBD remission patients, or healthy patients).

In other embodiments, comparing the relative protein expression levels of the biomarkers with a predetermined protein level may be used to assess if relapse of inflammatory bowel disease has occurred in the patient.

Moreover, the present application identifies novel biomarkers for determining if a subject with ulcerative colitis has pancolitis (where inflammation is present in the ascending colon) or non-pancolitis). These protein biomarkers are leukotriene A-4 hydrolase, thioredoxin domain containing protein 17, vasodilator-stimulated phosphoprotein, and thymosin beta-10.

In some embodiments, the relative protein expression levels of leukotriene A-4 hydrolase, thioredoxin domain containing protein 17, vasodilator-stimulated phosphoprotein, and thymosin beta-10 may be used to determine the presence of macro-inflammation in a patient.

Therefore, these biomarkers show different relative protein expression levels in gut samples from ulcerative colitis subjects with pancolitis when compared to corresponding protein expression levels from gut samples of subjects without pancolitis.

Measuring a single one of these biomarkers may be sufficiently reliable to indicate if an ulcerative colitis patient has or does not have pancolitis. However, it will be understood that measuring more than one of these biomarkers may increase the sensitivity and the specificity of the test. If all four or these biomarkers are used, the test may obtain an optimal level of specificity and sensitivity.

In some examples, these biomarkers may be used to assess the responsiveness of a patient with ulcerative colitis to treatment by monitoring the development or disappearance of pancolitis. A first sample may be taken at a first time (e.g. before treatment) to assess protein expression levels of the biomarkers as described herein to determine if the patient has or does not have pancolitis. A second sample may then be taken at a second time, analyzed for biomarker protein expression level as described herein, and compared to the results of the first sample. For instance, if the first sample indicated that the patient has pancolitis, but the second sample indicates instead that the patient no longer has pancolitis, then this may serve as an indication that inflammation has receded and that the patient may be responding to treatment. However, the results of the analysis made on the first sample may show instead that the patient has, for example, only a mild case of ulcerative colitis without pancolitis, but in the second sample it is determined that the patient has developed pancolitis, then this may be an indication that the patient may not be responding to treatment and that the patient's ulcerative colitis is in fact increasing in severity. The results from the test to establish responsiveness may also be correlated with a disease severity index to establish if the disease is receding, maintaining the same severity or worsening.

In other embodiments, comparing the relative protein expression levels of the biomarkers with a predetermined protein level may be used to assess if relapse of pancolitis has occurred.

### Definitions

In the present application, by subjects having “inflammatory bowel disease” it is meant subjects with ulcerative colitis (UC), subjects with Crohn's disease (CD) and/or subjects with IBD-unclassified (IBD-U).

By “gut sample” it is meant a sample that has originated from a subject's gastrointestinal track. In some examples, the gut sample may be specific to the colon. In other examples, the gut sample may be specific to the ascending colon or the descending colon. A gut sample may be a stool sample or any other non-invasive sample that has originated from the patient's gut. In some examples, the gut sample may involve obtaining a mucosal luminal interface (MLI) sample from the subject. In some examples, the gut sample may be a biopsy sample taken from a subject, obtained during, for instance, a colonoscopy.

By “subject” it is meant a pediatric subject and/or an adult subject. Even though the exemplary studies presented herein are conducted on pediatric subjects, there is considerable similarity in pathology between pediatric IBD and adult-onset IBD. The pediatric subjects of the exemplary studies were between eight and eighteen years of age. The age of the subjects did not have an impact on the results of the studies (i.e. the relative biomarker protein expression level). Therefore, it will be appreciated that similar relative biomarker protein expression levels may be observed in adult subjects, wherein the biomarkers may also be used, for instance, to detect IBD in an adult subject, or to determine if the adult subject has or does not have pancolitis.

By “severity of disease” it is meant if the disease is mild, moderate or severe (e.g. mild, moderate and severe may be defined in accordance with the Pediatric Ulcerative Colitis Activity Index, the Truelove and Witts' severity index, the Harvey-Bradshaw Index, or another diagnostic severity index). In some embodiments, “severity of the disease” may also mean determining whether the subject has an absence of disease (e.g. in some examples, indicating that the subject is in remission).

By “extent of disease” it is meant the proportion of the affected colon (i.e. the affected area), as defined, e.g., by the Paris Classification, where extent may be defined as E1, E2, E3 and E4.

By “measuring” a protein sample as used herein, it is meant conducting an analysis of a sample to determine a protein expression level (or relative protein expression level) using such techniques as an immunoassay (e.g. ELISA), semi-quantitative immunoblotting, mass spectrometry, and other techniques that are known in the art to quantitatively and/or qualitatively analyze the contents of a sample obtained from a patient.

Moreover, it will be understood that a protein name or its corresponding gene name may be used interchangeably herein to refer to the protein. Reference is made to Table 3 that correlates the different protein names with their respective gene names.

The Performed Study:

A quantitative proteomic analysis of the colonic MLI in treatment-naive pediatric IBD patients at two distinct colon sub-regions was performed. Analysis of the proteomic data identified candidate biomarkers that outperform the accuracy of calprotectin at the MLI for classification of IBD patients with colonic involvement compared with controls with normal appearing colons. In addition, biomarkers which can indicate extent of disease in UC (pancolitis vs non-pancolitis) were also identified. Finally, it is showed that the biomarker candidates LTA4H, Annexin A3 and CAT identified from the MLI proteome exhibited consistent results when assessed in stool samples.

Materials and Methods

Patient Cohort

A cross-sectional study of patients less than 18 years old undergoing diagnostic colonoscopy for IBD was performed. In order to assess the host proteomic landscape alterations in IBD, the following exclusion criteria were chosen as they are known to alter the intestinal microbiota and thus influence the host response: (1) body mass index>95th percentile; (2) diabetes mellitus (3) infectious gastroenteritis within the preceding 2 months; and (4) use of any antibiotics, probiotics or immunosuppressives within 1 month of colonoscopy. Moreover, patients with inconclusive IBD diagnosis at the time of sample collection were excluded from analysis.

It will be understood that even though the study focused on pediatric patients, the relative expression level of the identified biomarkers described herein may also be observed in adult patients to indicate the presence of IBD due to the significant overlap of disease etiology between pediatric IBD and adult IBD which results in similar gene and protein expression patterns.

Reference Standard Test Methods

IBD was diagnosed by clinical examination, endoscopy, imaging and laboratory testing³. The Pediatric Crohn's Disease Activity Index (PCDAI) was utilized for CD¹³ and the Pediatric Ulcerative Colitis Activity Index (PUCAI) was utilized for UC¹⁴. Inflammation of the mucosa of the ascending colon (AC) or descending colon (DC) was assessed by visual appearance at colonoscopy. Extent of macroscopically inflamed mucosa was classified using the Paris modification of the Montreal Classification for IBD¹⁵.

Colonic Mucosal-Luminal Interface (MLI) Aspirate Sample Collection

Colonic mucosal luminal interface (MLI) aspirates were obtained at time of diagnostic colonoscopy following a standard 1 day clean-out preparation¹⁶. Sampling occurred at the mid-ascending colon (AC) and/or at the site of the lower descending colon and upper sigmoid colon region (DC), and annotated to be from a normal, non-inflamed (CoN) or an affected, inflamed region (CoA) based on macroscopic evaluation. Briefly, upon insertion of the colonoscope, initial fluid and debris in the fluid were aspirated away. Thereafter sterile water was flushed onto the mucosa of the selected region and the fluid was aspirated into sterile collection vials as the MLI aspirate sample. These samples were used for analysis and the order of collection was distal to proximal sites. The samples were put on ice in the endoscopy room and immediately delivered to laboratory for further processing. A complete protease inhibitor cocktail (Roche Diagnostic GmbH, Mannheim, Germany) was added to the intestinal aspirates upon receipt in the lab. Following debris depletion by centrifugation at 700 g for 5 minutes at 4° C., the supernatant was subsequently subjected to 14,000 g centrifugation for 20 minutes at 4° C. to remove bacteria. The resultant supernatant was then filtered through a 0.2 μm syringe driven filter for removal of any residual bacterial cells and stored at −80° C.

Heavy Isotopic-Labeled Reference Proteome

Heavy reference proteins for quantification were prepared from 5 isotopically-labeled commercially available human cell lines, namely lymphocytic Jurkat (ATCC), HEK-293 (ATCC), colorectal carcinoma HCT 116 (ATCC), monocytic THP-1 (ATCC) and hepatic HuH-7 (JCRB Cell Bank). HCT116, HuH-7 and HEK293 cells were grown in custom prepared media¹⁷. THP-1 and Jurkat cells were grown in RPMI media (#0422 AthenaES Baltimore, Md., USA) supplemented with 15 mg/L methionine, 40 mg/L [13C6, 15N2]-L-lysine, 200 mg/L [13C6, 15N4]-L-arginine (Sigma Aldrich, Oakville, ON, Can), 10% dialyzed FBS (GIBCO-Invitrogen; Burlington, ON, CAN), 1 mM sodium pyruvate (Gibco-Invitrogen), 0.0059 g/L Phenol Red (Sigma Aldrich, Oakville, ON, Can) and 28 μg/mL gentamicin (Gibco-Invitrogen). Heavy amino acid incorporation (>95%) was confirmed by MS analysis¹⁸. For protein isolation, cells were lysed in lysis buffer (4% SDS, 50 mM Tris, pH 8.0, protease inhibitor cocktail (Roche Diagnostic GmbH, Mannheim, Germany)) and sonicated three times in 10 second pulses with a 30 second incubation on ice between pulse intervals. Lysates were centrifuged at 10,000 g for 10 minutes, supernatants collected and protein concentrations were quantified by DC (detergent compatible) protein assay (BIORAD, California, USA). Protein aliquots were stored at −80° C.

Processing of Colonic MLI Aspirate Proteins for Mass Spectrometry Analysis

Filtered colonic MLI aspirates were thawed on ice, and proteins precipitated overnight with trichloroacetic acid (20% v/v). Protein pellets were washed three times with acetone (100%) and dried before resuspension and sonication in lysis buffer. Protein concentration was quantified by DC protein assay (BIORAD, California, USA). Colonic aspirate proteins (45 μg) were combined with an equal amount of heavy isotopic-labeled cell lysate (9 μg of each cell type), used as a representative internal standard to permit quantitative proteomic data, an approach known as Super-SILAC (stable-isotope labeling by amino acids in cell culture)¹⁹. The mixture containing proteins from colonic aspirates and internal reference cells was digested with trypsin by filter aided sample preparation method (FASP)²⁰, fractionated into 5 fractions (pH 4, 6, 8, 10 and 12) using SCX resin (Agilent Technologies, CA, USA), and desalted with a 10 μm AQUA-C18 resin (Dr Maisch, GmbH, Ammerbuch, Germany).

LC-MS/MS and Bioinformatic Analysis

High-performance liquid chromatography/electrospray ionization tandem mass spectrometry (HPLC-ESI-MS/MS) was performed¹² using an Ekspert nanoLC 400 (Eksigent, Dublin, Calif., USA) coupled to an LTQ Velos Pro Orbitrap Elite MS (ThermoFisher Scientific, San Jose, Calif.).

Peptides were assigned and quantified using MaxQuant version 1.5.3.30 21 in a single run against the human Uniprot database (downloaded 2012 Jul. 11). Patients with inconclusive IBD diagnosis at the time of sample collection were excluded from downstream bioinformatics analysis. The following parameters were used: a multiplicity of two with Arg10 and Lys8 selected as the heavy labels; a specific digestion mode was implemented with trypsin selected as the enzyme with a maximum of two missed cleavages; cysteine carbamidomethylation as a fixed modification; methionine oxidation and acetylation (protein N-termini) as variable modifications; the re-quantify and match between runs parameters were enabled; minimum peptide length of seven amino acids; ion mass tolerance of 0.5 Da; protein and peptide false discovery rate (FDR) of 1%. The AC and DC proteomes were analyzed separately post database search.

The MaxQuant output (Ratio H/L normalized) was uploaded into Perseus v1.5.2.6 for heavy/light inversion, log 2 transformation, Pearson correlation coefficient determination and hierarchical clustering of correlation values, while data filtering was performed in Excel. Partial least squares discriminant analysis (PLS-DA) and receiver operating characteristics (ROC) curve analyses were performed on k-nearest neighbor (knn) imputed data in the Biomarker Analysis module of MetaboAnalyst 3.022. Using predictive class probability values generated in MetaboAnalyst 3.0, ROC curves and predictive class probability plots were plotted in GraphPad Prism 7 and ROC curve AUC calculated in GraphPad Prism 7. ROC, sensitivity and specificity confidence intervals were calculated in GraphPad Prism 7 using predictive class probability values generated in MetaboAnalyst 3.0. Individual protein AUC and associated confidence interval values were calculated using knn imputed MS results in GraphPad Prism7. PCA was performed using the prcomp argument in R studio. Protein interaction networks were generated using STRING version 10.0 and Cytoscape version 3.4.0. For suspected pediatric IBD biomarker panel generation, proteins identified by PLS-DA in both the AC and DC with the highest combined area under the curve (AUC) upon ROC curve analysis were considered, and biomarker panel assembled based on iterative analysis. If a protein lead to a decrease in sensitivity or specificity without an increase in its counterpart in either colon sub-region it was skipped. Biomarker panel generation for extent of disease in UC considered features with the highest AUC from comparison of ascending colon with and without macroscopic evidence of inflammation. Relative protein expression graphs with statistical analyses were generated in GraphPad Prism 7. Biomarker panels and Calprotectin (S100-A8 & S100-A9) were evaluated using the ROC curve based model evaluation module of MetaboAnalyst 3.0. Gene ontology was performed using DAVID Bioinformatics Resources 6.823 and plotted in GraphPad Prism 7.

Study data were collected and managed using REDCap electronic data capture tools hosted at the CHEO Research Institute. REDCap (Research Electronic Data Capture)²⁴.

Stool Collection and Protein Extraction from Stool

Stool samples were collected from a treatment-naive cohort consisting of both independent samples from the discovery cohort and patients whose corresponding MLI sample was used for biomarker discovery. Stool samples were collected within 8 weeks of diagnostic colonoscopy with 82.2% collected within 4 days of colonoscopy. Stool samples were frozen after collection and brought into the clinic on ice. Extraction buffer (50 mM Tris, pH 7.2, 150 mM NaCl, protease inhibitor cocktail (Complete, Mini (Roche Diagnostic GmbH, Mannheim, Germany)) was added to stool in a 5:1 ratio (extraction buffer volume:stool weight). The resultant slurry was mixed by agitation for 30 seconds, followed by rotation at 4° C. for 20 minutes. Following centrifugation for 20 minutes at 10,000 g at 4° C. to remove large sediment, the supernatant was filtered (0.2 μm) and the protein-containing filtrate was collected and stored at −80° C. Protein concentration was assessed by BCA protein assay kit (ThermoFisher Scientific, San Jose, Calif.).

Immunoblotting

Proteins from intestinal aspirates were precipitated, lysed and quantified. Intestinal aspirate proteins (30 μg) or stool protein extracts (50 μg) were separated by a 4-15% TGX Stain-free gel (BIORAD, California, USA) under reducing conditions. The gels were exposed to UV light for 1 minute to enable fluorescent labeling of proteins, followed by electroblotting to LF PVDF membranes (BIORAD, California, USA) using the Trans-Blot Turbo Transfer system (BIORAD, California, USA), followed by another round of UV exposure. Standard immunoblotting procedures were applied, using antibodies for Annexin A3 (ab33068 Abcam Ltd, Cambridge, England) with anti-rabbit secondary antibody (GE Healthcare Life Sciences, Massachusetts, USA). Proteins were detected by ECL substrate (BIORAD, California, USA) and blots were imaged using the ChemiDoc MP system (BIORAD, California, USA). Quantification was performed using Image Lab 5.2.1 (BIORAD, California, USA) utilizing the full lane intensity as reference for normalization.

Enzyme-Linked Immunosorbent Assay (ELISA)

Leukotriene A-4 hydrolase (abx572445 Abbexa Ltd, Cambridge, UK) and Catalase (ab171572 Abcam, Cambridge, UK) from stool samples was measured from 50 ug and 0.5 μg of protein, respectively, by ELISA which was then performed according to the manufacturer's protocol. A reference standard was added to all plates for inter-plate normalization.

Results

Patient Cohort

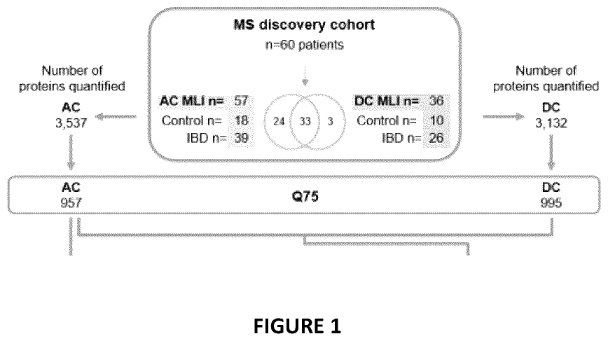

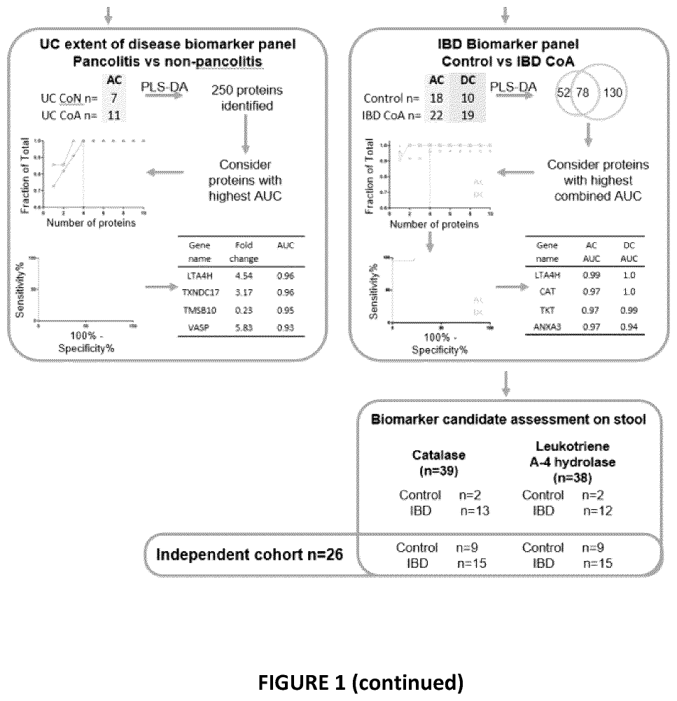

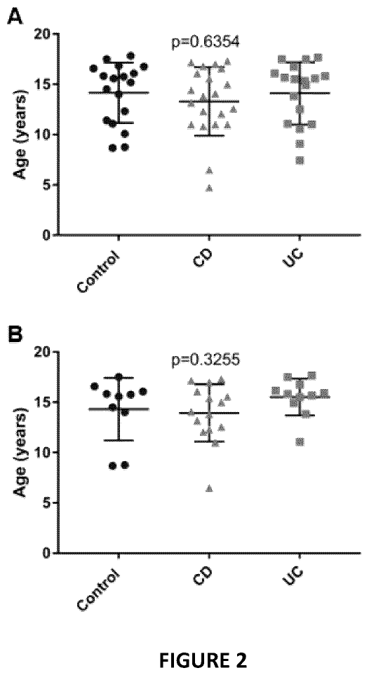

The colonic MLI aspirates of 93 colon sub-regions (57 AC [18 control, 39 IBD], 36 DC [10 control, 26 IBD]) were collected during diagnostic endoscopy from 60 patients prior to the administration of any treatment. For 33 patients (control=10, CD=14, UC=9) both colon sub-regions were analyzed, whereas the AC or DC proteomes were exclusively analyzed for 24 (control=8, CD=7, UC=9) and 3 (CD=1, UC=2) patients, respectively (FIG. 1). The patient characteristics are summarized in Table 1. No significant difference in age was observed between groups in either colon sub-region (supplementary FIGS. 1A, B) or between sexes in the IBD group of the AC and DC and the control group of the DC, although a significantly higher number of females in the AC control group was observed (Binomial one-tailed; p=0.0481). In accordance with previous reports, the majority (70%) of pediatric UC patients in the cohort presented with extensive disease (Paris E3/E4 classification)²⁵. Similarly, the majority (77%) of CD patients had disease involvement that was either isolated to the colon (L2) or had ileocolonic (L3) involvement²⁶.

TABLE 1
Patient characteristics (AC = ascending colon; DC = descending colon)

| | Controls (n = 18) | | CD (n = 22) | | UC (n = 20) | |
|---|---|---|---|---|---|---|
| Region | AC (n = 18) | DC (n = 10) | AC (n = 21) | DC (n = 15) | AC (n = 18) | DC (n = 11) |
| # Females (%) | 13 (72.2%) | 7 (70.0%) | 5 (23.8%) | 3 (20.0%) | 18 (55.6%) | 4 (36.4%) |
| Median Age, years | 15.4 | 15.7 | 13.8 | 14.1 | 15.4 | 15.8 |
| (IQR) | (11.3-16.6) | (12.7-16.2) | (11-16.3) | (12.3-16.1) | (11.1-16.2) | (14.9-16.8) |
| Paris Classification | | | | | | |
| A1a | NA | NA | 4 (19.0%) | 2 (13.3%) | NA | NA |
| A1b | NA | NA | 16 (76.2%) | 12 (80.0%) | NA | NA |
| A2 | NA | NA | 1 (4.8%) | 1 (6.7%) | NA | NA |
| Location | | | | | | |
| L1 | NA | NA | 4 (19.0%) | 3 (20.0%) | NA | NA |
| L2 | NA | NA | 5 (23.8%) | 4 (26.7%) | NA | NA |
| L3 | NA | NA | 11 (52.4%) | 7 (46.7%) | NA | NA |
| L4a | NA | NA | 10 (46.6%) | 6 (40.0%) | NA | NA |
| L4b | NA | NA | 3 (14.3%) | 3 (20.0%) | NA | NA |
| Behavior | | | | | | |
| B1 | NA | NA | 21 (100%) | 14 (93.3%) | NA | NA |
| B2 | NA | NA | 0 | 0 | NA | NA |
| B3 | NA | NA | 0 | 1 (6.7%) | NA | NA |
| P | NA | NA | 3 (14.3%) | 5 (33.3%) | NA | NA |
| Growth | | | | | | |
| G0 | NA | NA | 14 (66.7%) | 11 (73.3%) | NA | NA |
| G1 | NA | NA | 7 (33.3%) | 4 (26.7%) | NA | NA |
| Extent | | | | | | |
| E1 | NA | NA | NA | NA | 1 (5.6%) | 1 (9.1%) |
| E2 | NA | NA | NA | NA | 4 (22.2%) | 4 (36.4%) |
| E3 | NA | NA | NA | NA | 2 (11.1%) | 2 (18.2%) |
| E4 | NA | NA | NA | NA | 11 (61.1%) | 4 (36.4%) |
| Severity | | | | | | |
| S0 | NA | NA | NA | NA | 15 (83.3%) | 9 (81.8%) |
| S1 | NA | NA | NA | NA | 3 (16.7%) | 2 (18.2%) |

Proteomic Dataset Assessment

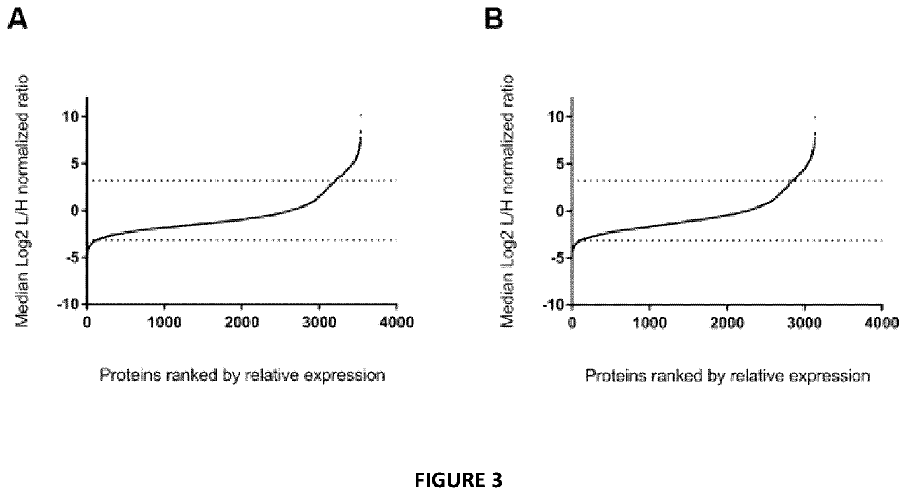

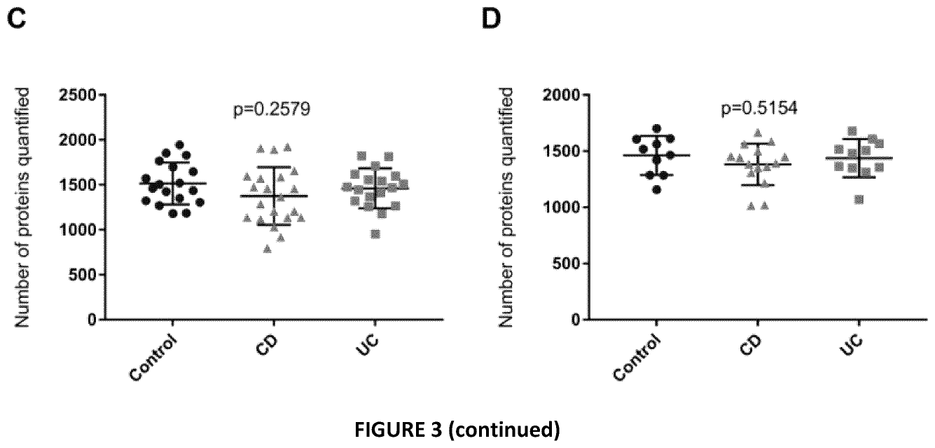

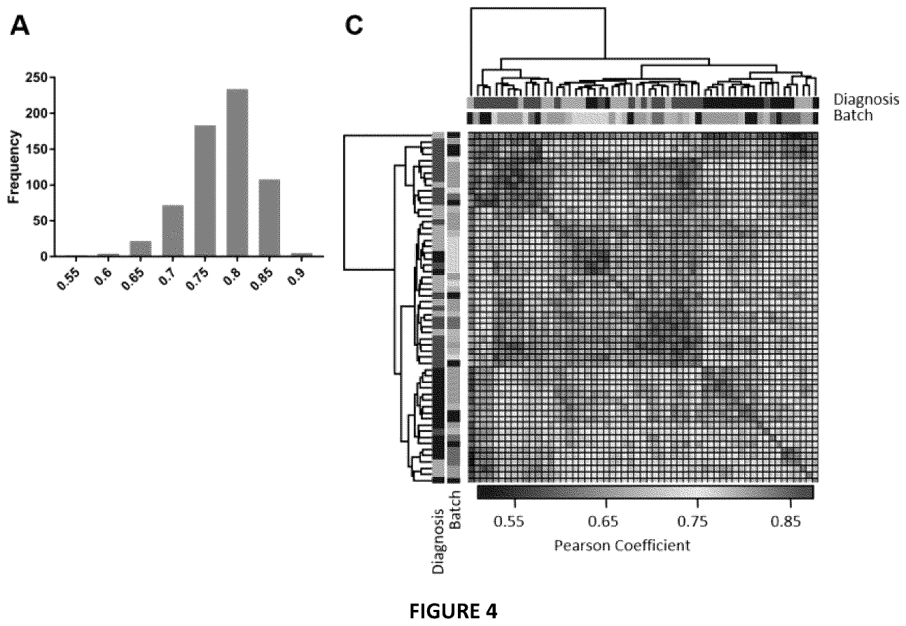

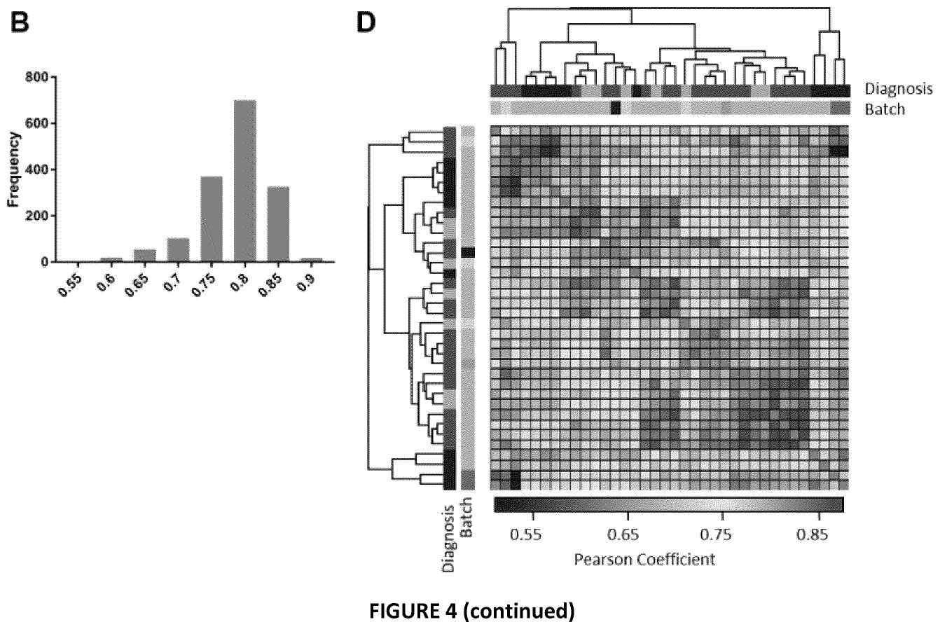

Samples were processed and analyzed by MS over a period of 22 months. A super-SILAC approach was implemented for accurate quantification of proteins obtained from colonic MLI aspirate samples, and for use as a consistent reference over time. The majority (87.7% (AC), 88.5% (DC)) of proteins were quantified within a 10-fold median ratio (normalized MLI proteins/super-SILAC reference proteome). Furthermore, there were no significant differences observed in the total number of quantified proteins between groups in either colon sub-region. A total of 3,537 and 3,132 proteins were quantified from the AC and DC MLI, respectively, of which 972 (AC) and 995 (DC) proteins were quantified in 75% of samples (Q75) with 80% protein ID overlap between regions. Pearson correlation analysis of the Q75 was performed on the log 2 light/heavy ratios (FIGS. 3A and 3B), yielding 89% and 84% of values with a correlation>0.75 in the AC and DC respectively, indicating consistent MS performance, sample preparation and that the majority of the proteome remains unaltered in the disease state. Hierarchical clustering of the Pearson correlation values tended to segregate samples according to diagnosis and inflammatory status rather than by MS batch analysis (FIGS. 4C and 4D).

Proteomic Landscape Alterations in Treatment Naïve Pediatric IBD

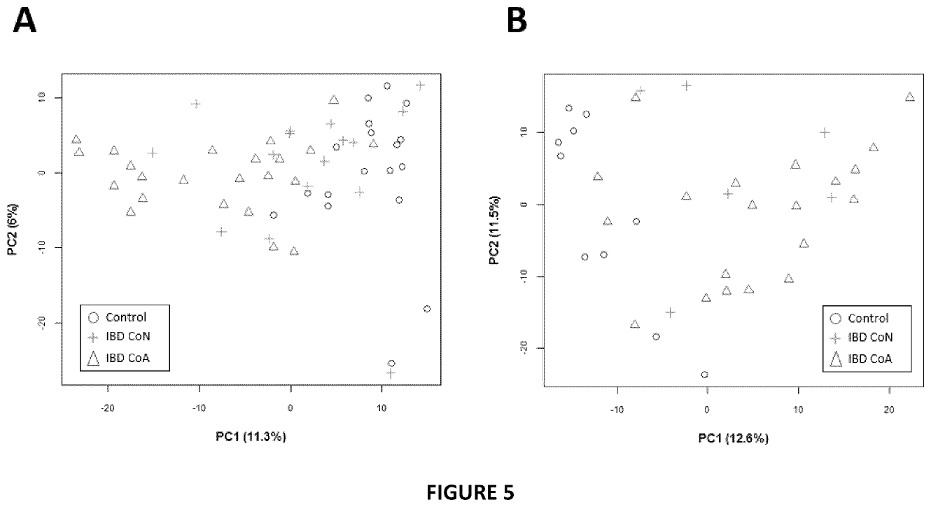

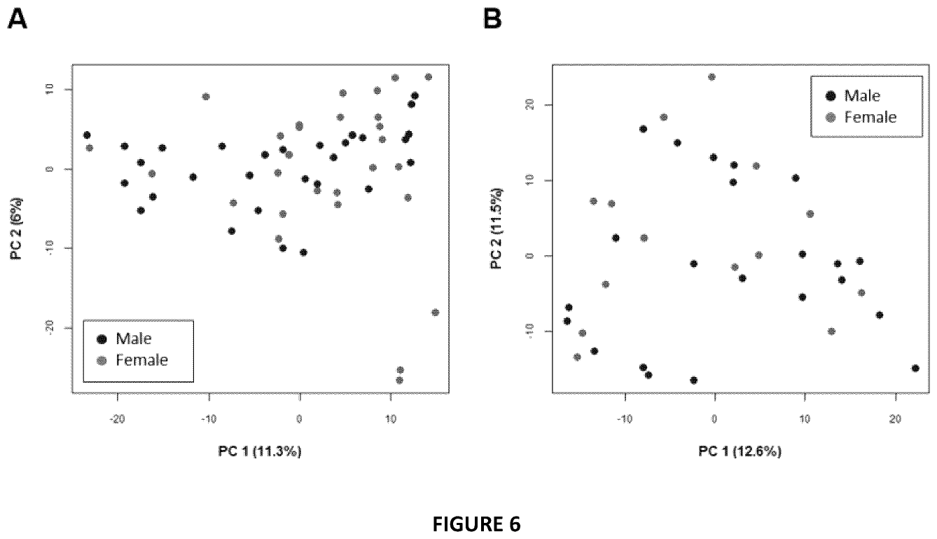

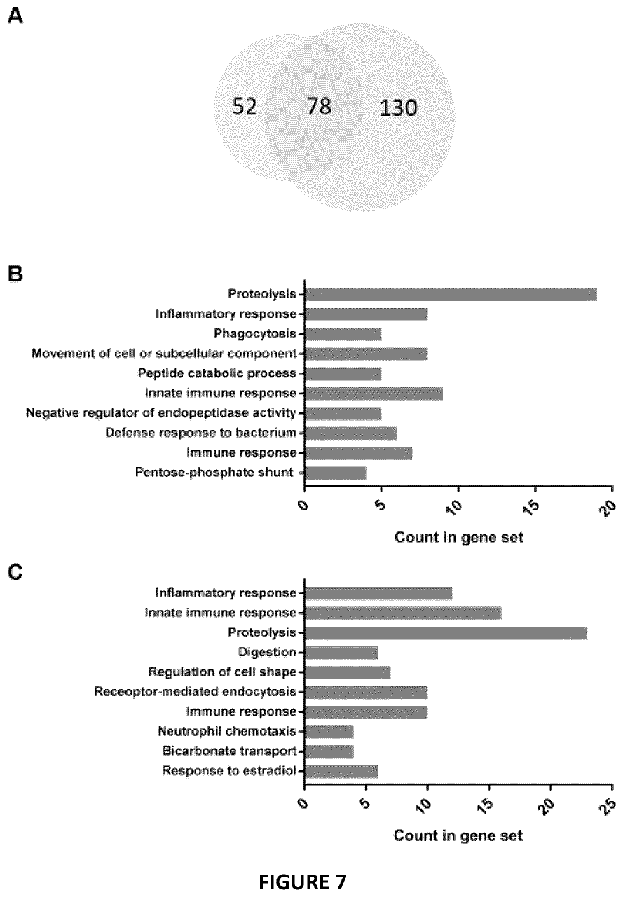

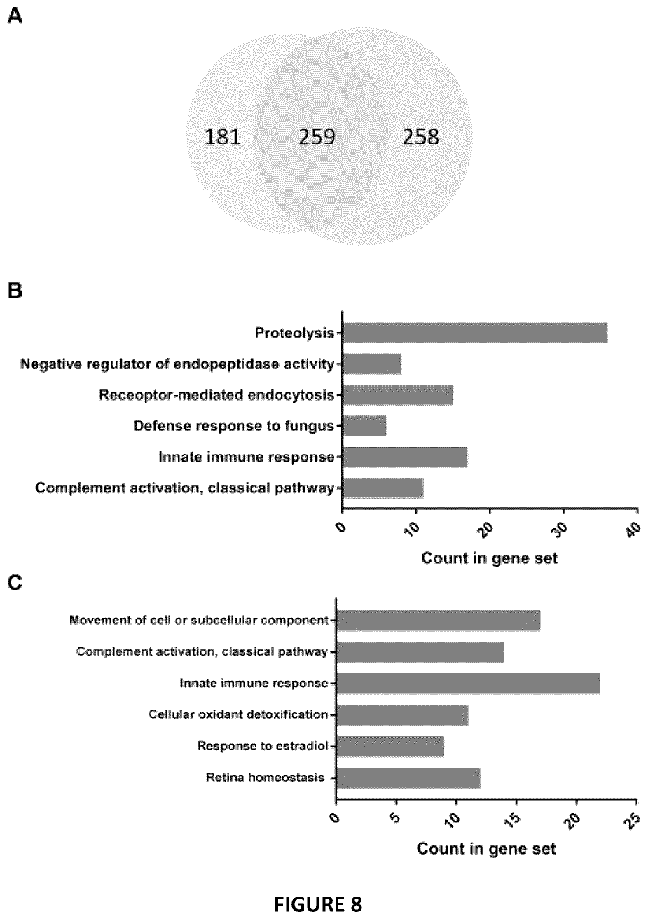

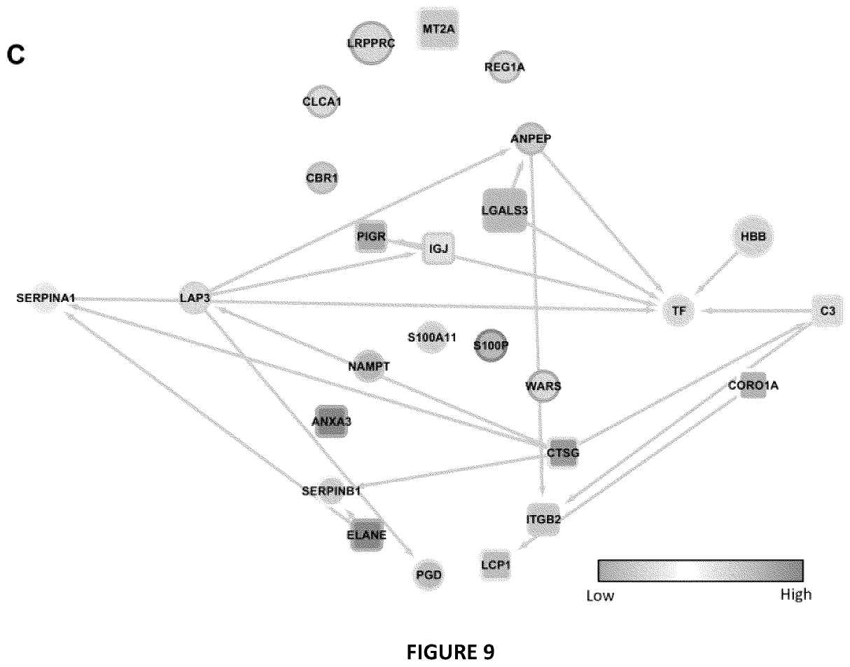

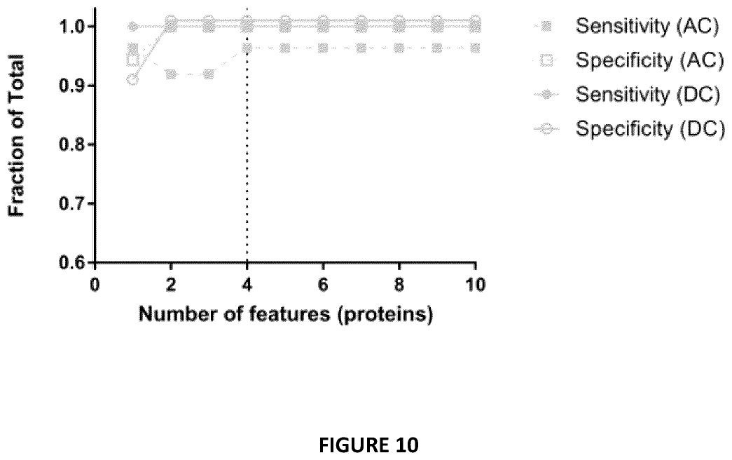

To assess the proteomic alterations in an unbiased manner, principal component analysis (PCA) was performed on the Q75. The control samples generally clustered separately from those IBD samples that were deemed by endoscopy to have macroscopic evidence of inflammation (IBD CoA) (FIG. 5). In the AC, the IBD samples with no evidence of macroscopic inflammation (IBD CoN) distributed between control samples and IBD CoA samples, whereas in the DC the IBD CoN samples tended to cluster with the IBD CoA. Segregation according to sex was not observed in either colon sub-regions (FIG. 6). In order to identify discriminate features between control patients and IBD patients with active disease (IBD CoA), a multivariate analysis approach using PLS-DA was applied, identifying 130 and 208 proteins in the AC and DC respectively. There were 78 proteins common to both the AC and DC (FIG. 7A), which included proteins known to be altered in IBD such as the S100-A8 subunit of calprotectin. Gene ontology enrichment of the PLS-DA results yielded several expected biological processes related to IBD including defense response to bacterium, innate immune response, inflammatory response, phagocytosis and neutrophil chemotaxis (FIGS. 7B and 7C). Upon PLS-DA analysis of controls and IBD samples without macroscopic evidence of inflammation (CoN), pathways related to inflammation were observed (FIG. 8), possibly indicating the presence of microscopic inflammation. Notably, of the 130 proteins in the current AC IBD CoA vs control data set, 26 were also identified¹² (FIG. 9). AC biopsies were analyzed, wherein 225 proteins were identified as discriminant features of IBD CoA vs control by PLS-DA analysis of the discovery cohort. Among these 26 common proteins, 11 (42%) and 9 (35%) were increased and decreased, respectively, in both datasets. The remaining 6 (23%) common proteins exhibited opposite relative expression between the biopsy and MLI datasets, with the majority (4/6) being elevated in the IBD samples relative to control in the MLI proteome versus a relative reduction in the biopsy proteome. Within these 26 proteins, 22 localize to the extracellular region and an enrichment of proteins related to the immune response was identified (11/26). The expression levels of the 26 proteins in biopsy samples versus mucosal luminal interface samples are listed in Table 2:

TABLE 2
the expression levels of 26 proteins in MLI samples and biopsy samples,
showing opposite relative expression for 6 of the proteins.

| | MLI expression | Biopsy |
|---|---|---|
| | ratio log 2 | expression |
| Gene | of IBD | of log2 (IBD |
| name | CoA/Control AC | CoA/Control) |
| ANPEP | −1.98915 | −0.6181 |
| ANXA3 | 4.216674 | 1.563199 |
| C3 | 1.356244 | −0.24447 |
| CBR1 | −2.0791 | −0.57351 |
| CLCA1 | −1.33476 | −0.60407 |
| CORO1A | 2.680741 | −0.01155 |
| CTSG | 3.547383 | 0.604387 |
| ELANE | 4.154011 | 1.545775 |
| HBB | 1.602313 | −0.20574 |
| IGJ | −1.09184 | −0.4301 |
| ITGB2 | 2.013377 | 0.941676 |
| LAP3 | −1.4226 | 0.89655 |
| LCP1 | 2.330294 | 0.539129 |
| LGALS3 | −2.6157 | −0.54731 |
| LRPPRC | −1.58638 | −0.74149 |
| MT2A | −2.0682 | −0.2031 |
| NAMPT | 2.595841 | 1.007 |
| PGD | 2.369553 | 0.653318 |
| PIGR | −3.22623 | −0.43575 |
| REG1A | −1.48006 | −0.63364 |
| S100A11 | 1.800538 | 0.79001 |
| S100P | 2.540318 | 2.302368 |
| SERPINA1 | −0.91455 | 0.315549 |
| SERPINB1 | 1.7999 | 0.254763 |
| TF | 1.438568 | −0.26176 |
| WARS | 1.39081 | 1.680909 |

Biomarker Panel for Suspected Pediatric IBD Diagnosis

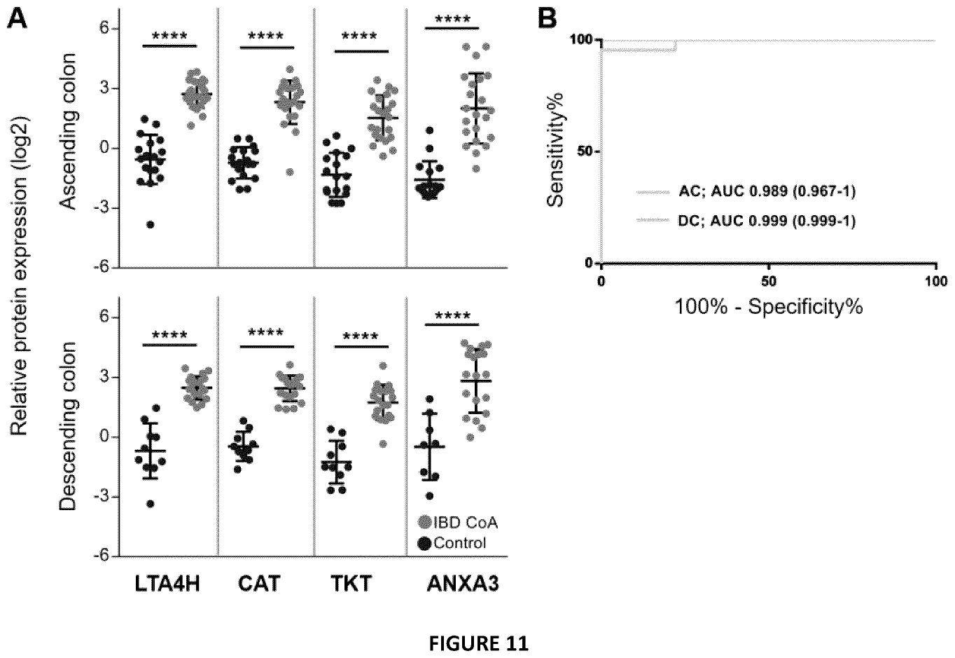

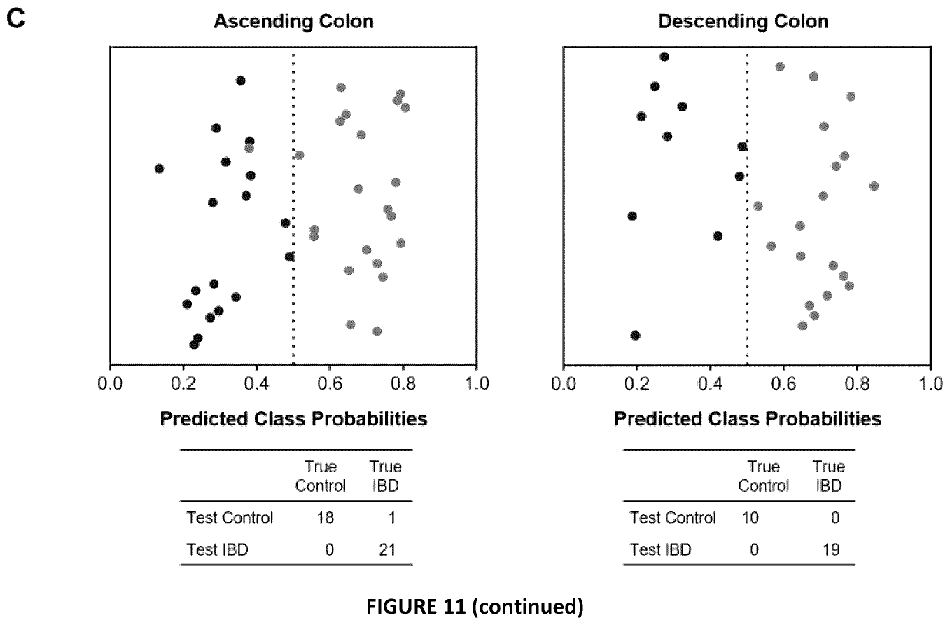

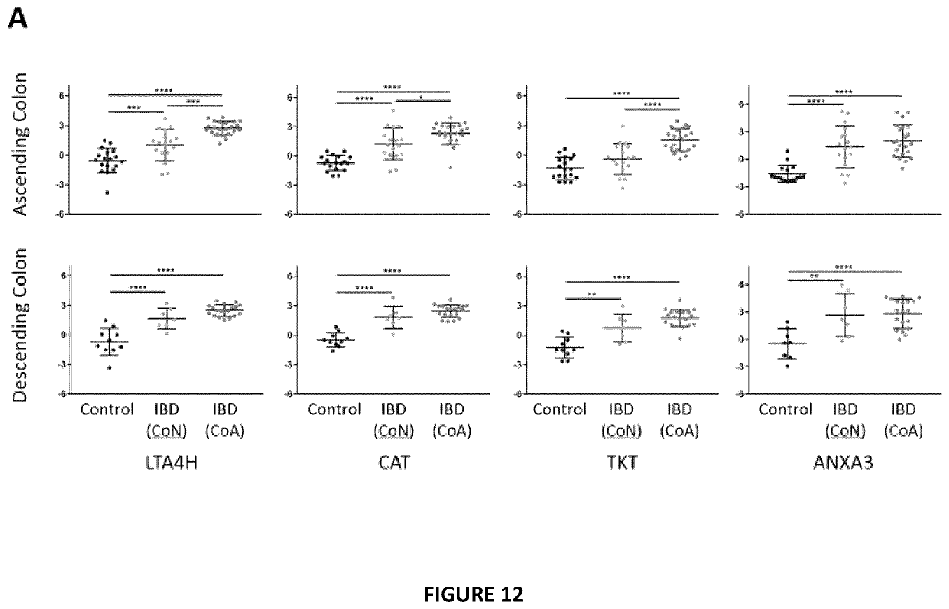

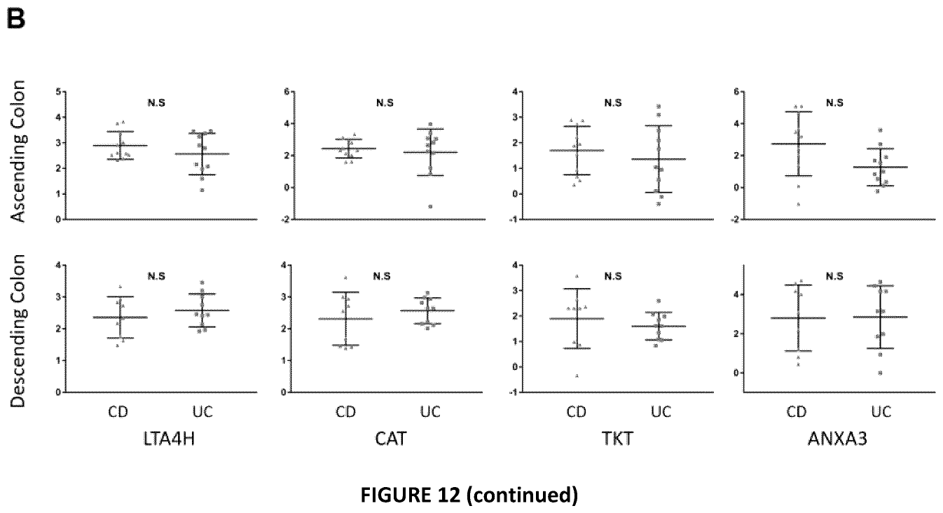

To identify biomarkers of active IBD, discriminant features identified by PLS-DA (control vs IBD CoA) were further considered for biomarker panel generation. Proteins with the highest combined area under the curve (AUC) values from both colon sub-regions were considered for biomarker panel assembly. A maximum sensitivity and specificity utilizing the minimum number of features was reached in the AC using a panel of 4 features, whereas 2 features were sufficient in the DC to reach maximum sensitivity and specificity; the 4 panel proteins ultimately utilized for IBD CoA vs Control are listed in Table 3. The relative expression levels are depicted in FIG. 11 and compared to that of IBD CoN in supplemental FIG. 12. While not included in the development of the panel, IBD CoN levels of all panel proteins were significantly different from control samples with the exception of transketolase in the AC. However, it is observed that the relative expression level of these biomarkers in subjects with IBD CoN are higher than those for the controls. Therefore, a skilled person will understand that these biomarkers may also be used to detect the presence of IBD CoN in a subject.

TABLE 3
Proteins in panel for pediatric IBD diagnosis and for extent of disease in
UC (pancolitis vs non-pancolitis)

| | | | | | | Fold | AUC |
|---|---|---|---|---|---|---|---|
| | | Fold change | | AUC | | change | (CI) |
| | | [IBD | | (CI) | | [UC CoA/ | [UC CoN vs |
| | Gene | CoA/Ctrl] | | [IBD CoA vs Control] | | UC CoN] | UC CoA] |
| Protein | name | AC | DC | AC | DC | AC | AC |
| Leukotriene A-4 | LTA4H | 8.05 | 6.66 | 0.995 | 1.0 | 4.54 | 0.961 |
| hydrolase | | | | (0.982-1.008) | (1.0-1.0) | | (0.881-1.041) |
| Catalase | CAT | 8.83 | 7.31 | 0.967 | 1.0 | N/A | N/A |
| | | | | (0.903-1.031) | (1.0-1.0) | | |
| Transketolase | TKT | 7.23 | 7.39 | 0.967 | 0.989 | N/A | N/A |
| | | | | (0.922-1.012) | (0.962-1.017) | | |
| Annexin A3 | ANXA3 | 18.59 | 9.35 | 0.972 | 0.937 | N/A | N/A |
| | | | | (0.929-1.015) | (0.849-1.025) | | |
| Thioredoxin | TXNDC17 | N/A | N/A | N/A | N/A | 3.17 | 0.961 |
| domain-containing | | | | | | | (0.881-1.041) |
| protein 17 | | | | | | | |
| Thymosin | TMSB10 | N/A | N/A | N/A | N/A | 0.23 | 0.948 |
| beta-10 | | | | | | | (0.851-1.045) |
| Vasodilator- | VASP | N/A | N/A | N/A | N/A | 5.83 | 0.935 |
| stimulated | | | | | | | (0.824-1.046) |
| phosphoprotein | | | | | | | |

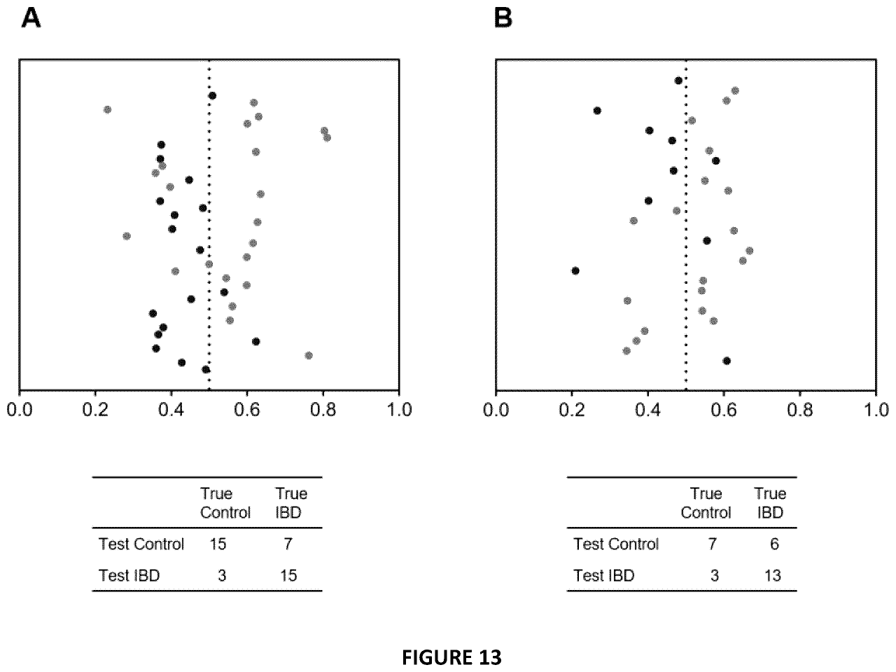

Applying a receiver operating characteristics (ROC) curve utilizing this panel of 4 proteins achieves an AUC value of 0.989 (95% CI: 0.967-1.0) and 0.999 (95% CI: 0.999-1.0) for the AC and DC, respectively (FIG. 11B). Predictive class probabilities yielded a sensitivity of 0.954 (95% CI: 0.7716-0.9988) and 1.0 (95% CI: 0.8235-1.0) for the AC and DC, respectively and a specificity of >0.999 (AC 95% CI: 0.8147-1.0; DC 95% CI: 0.6915-1.0) for both the AC and DC, providing a classification accuracy of 97.5% and 100% in the AC and DC, respectively. This panel of biomarkers for suspected pediatric IBD diagnosis outperformed the classification accuracy of calprotectin (S100-A8 & S100-A9) by a direct comparison of MS data obtained from the MLI in both colon sub-regions. Calprotectin yielded a sensitivity of 0.682 (95% CI: 0.4513-0.8614) and 0.684 (95% CI: 0.4345-0.8742) for the AC and DC respectively, and a specificity of 0.833 (95% CI: 0.5858-0.9642) and 0.700 (95% CI: 0.3475-0.9333) in the AC and DC, respectively, which yielded classification accuracies of 75.0% and 69.0% in the AC and DC, respectively (FIG. 13).

Biomarker Panel for Extent of Disease in UC (Pancolitis Vs Non-Pancolitis)

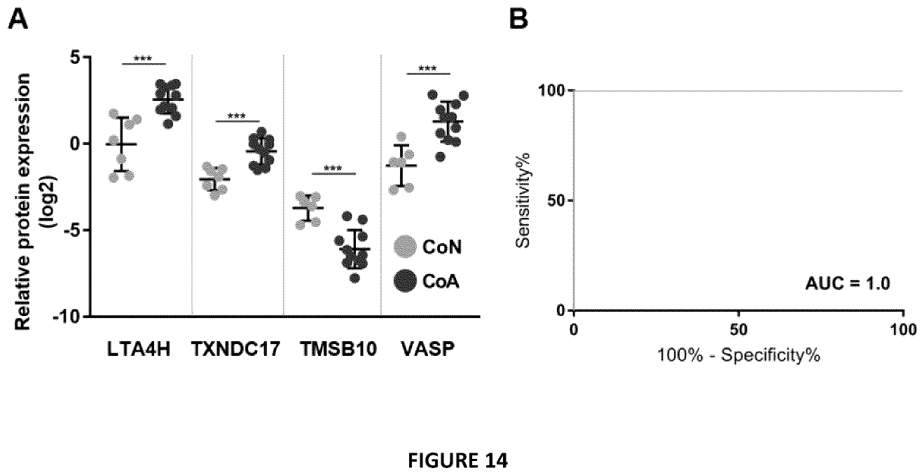

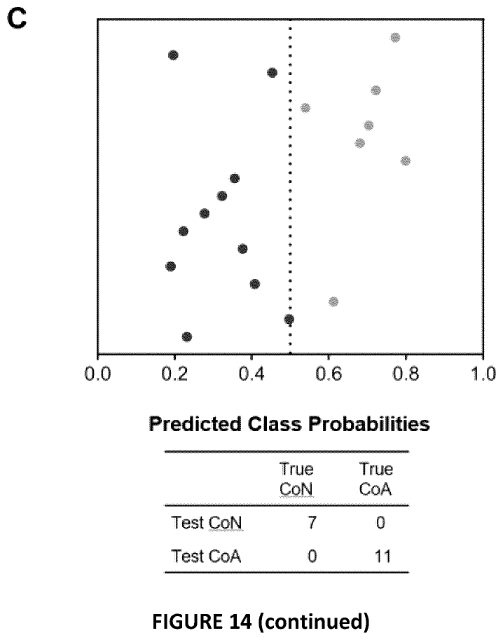

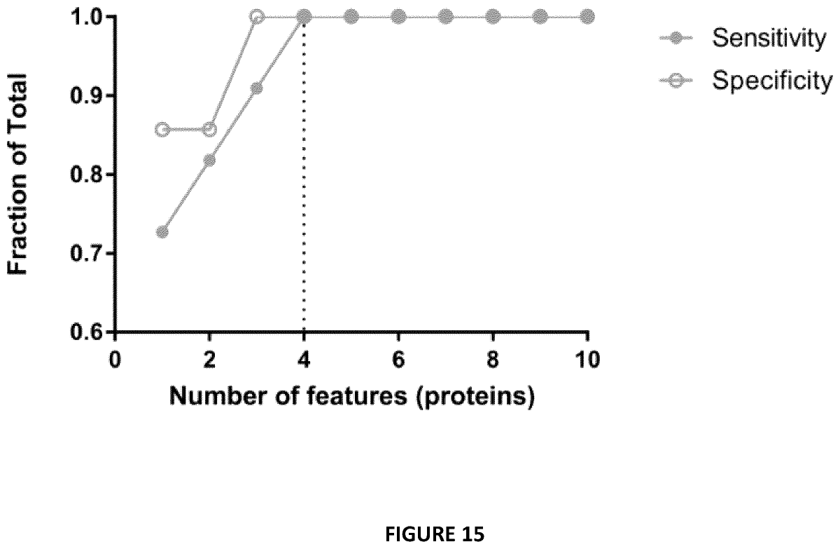

The majority (70%) of new-onset pediatric UC patients recruited in the discovery cohort displayed extensive disease (E3 or E4). To generate a biomarker panel to evaluate disease extent, features identified by PLS-DA upon comparison of colonic aspirate proteins arising from the AC of patients with evidence of macroscopic inflammation (UC CoA) versus proteins from the AC of patients without evidence of macroscopic inflammation (UC CoN) were further considered. As listed in Table 3, a panel of 4 proteins achieved a sensitivity of >0.9999 (95% CI: 0.5904-1.0) and specificity of >0.9999 (95% CI: 0.7151-1.0) and yielded a classification accuracy of 100% (FIGS. 14 and 15).

Validation of Biomarkers Identified at the MLI in Stool Samples

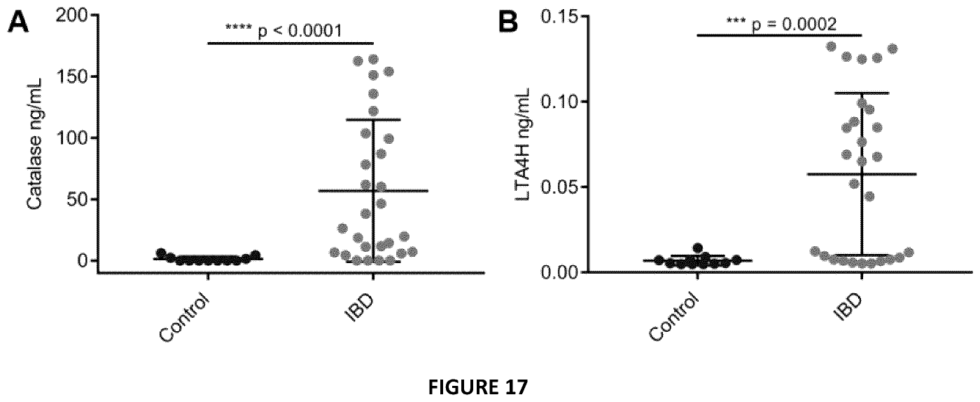

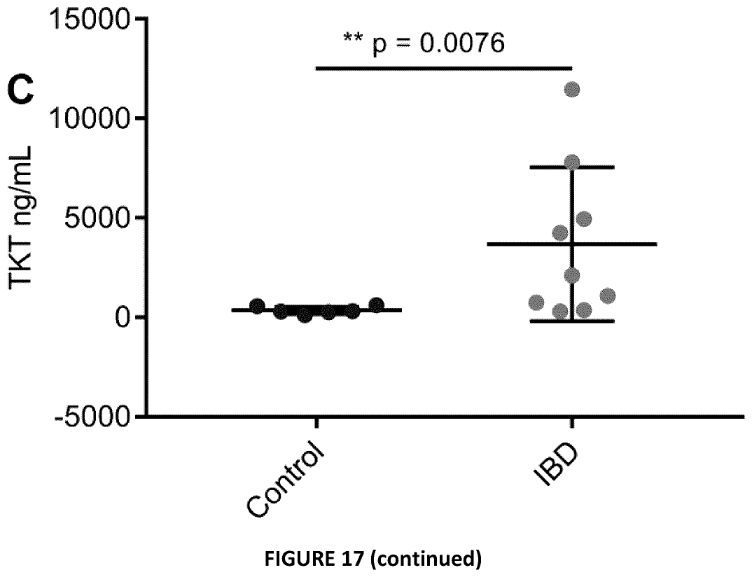

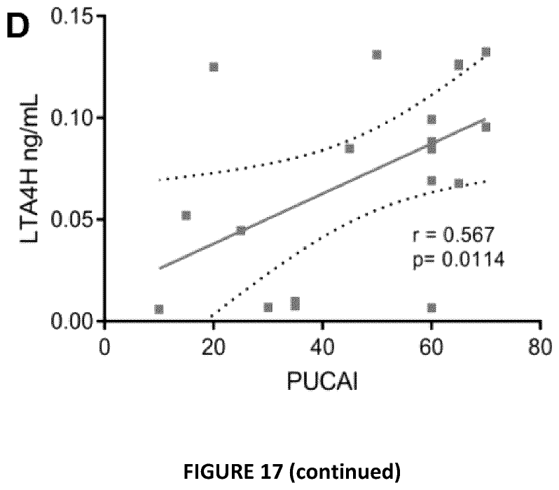

In order to implement easily accessible biomarkers that would be more readily translatable to the clinic, the expression of 2 of the IBD biomarker proteins identified herein, namely CAT and LTA4H, was assessed in stool. ELISA was performed on stool from 39 and 38 children for CAT and LTA4H, respectively. This cohort encompasses both independent participants (n=26) and participants for which their MLI samples were utilized for biomarker discovery (n=15). The increased expression in IBD compared to control observed by proteomics at the MLI was reflected by ELISA in stool, in which the expression of CAT (p<0.0001), LTA4H (p=0.0002) and transketolase (p=0.0076) were significantly different in IBD patients compared to controls (FIGS. 17A, 17B and 17C). Furthermore, the expression of LTA4H in stool correlated with the PUCAI (Spearman r=0.567 (95% CI: 0.1378-0.817; p=0.0114) (FIG. 17D). LTA4H also correlated with albumin (r=−0.3696; p=0.0265) but did not correlate with ESR or HCT, which are used in the calculation of the PCDAI, nor with PCDAI. The expression of CAT in stool correlated with the level of albumin measured in serum (r=−0.4645; p=0.0038) but not with PCDAI, PUCAI, HCT or ESR.

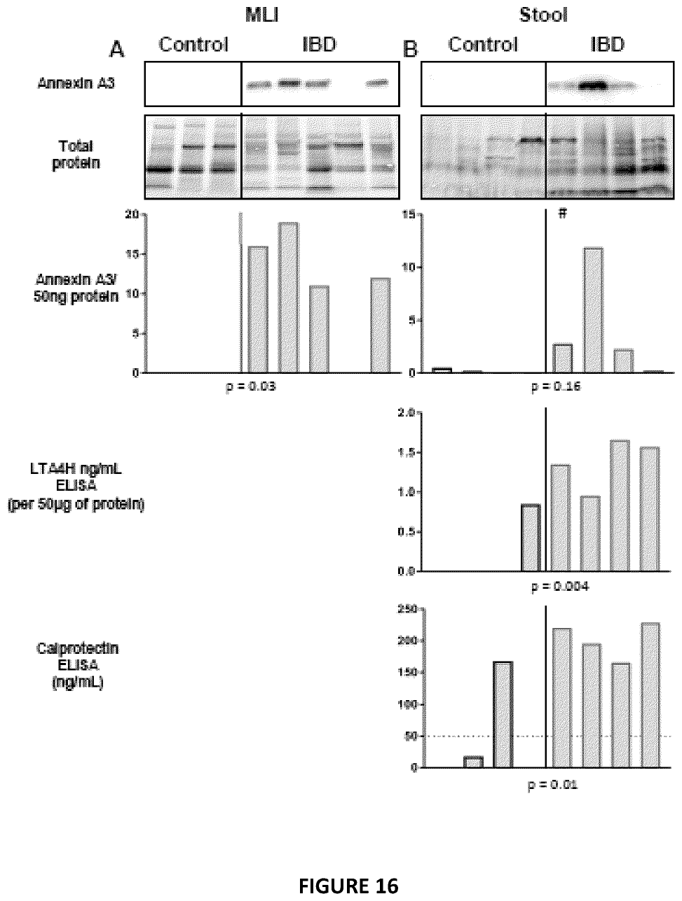

The increased expression of Annexin A3 in IBD patients with active disease compared to the controls was validated by immunoblotting using DC MLI samples independent of the discovery cohort (FIG. 16A). Quantitation of immunoblots are shown relative to total protein. To investigate whether Annexin A3 could be detected in a non-invasive bio-specimen, and thus more readily translatable to the clinic, immunoblotting on stool samples from patients included in the discovery cohort in addition to independent samples was performed, wherein a single patient had commenced therapy upon stool collection. In accordance with the MLI samples, Annexin A3 was detected in the IBD stool samples (FIG. 16B). For direct comparison, the level of fecal calprotectin in the same samples was measured using an ELISA kit that has been applied in the clinical setting. Notably, one control patient had levels of fecal calprotectin considered to be positive for IBD diagnosis. Therefore, the calprotectin results yielded a false positive IBD result for a given patient. In contrast, the immunoblots correctly identified all control patients as non-IBD, this determination based upon the relative level of Annexin A3 (FIG. 16B).

Moreover, the levels of LTA4H were evaluated by ELISA from the same stool samples as calprotectin and Annexin A3. There was a significant increase (**p=0.004) in the levels of LTA4H in stool samples obtained from IBD patients compared to control patients (FIG. 16C). Furthermore, the expression level of LTA4H in the stool samples correctly classified all the patients as either controls or IBD, in contrast to when using the calprotectin results.

Discussion of the Results

The study identified biomarkers that improve upon current IBD standards in diagnosis, and propose novel biomarkers for disease extent in UC. Biomarkers currently applied in the clinic, including fecal calprotectin, are limited in their ability to diagnose and monitor IBD, and thus can only be successfully used when in conjunction with endoscopy.

An IBD biomarker panel of four proteins was identified that was capable of accurately classifying >95% of patients as IBD or control using MLI aspirates. One or more of these biomarkers may also be used to reliably classify patients as IBD-positive or IBD-negative. Furthermore, it was shown that the significantly different levels of select biomarker panel proteins were confirmed in a non-invasive bio-specimen (stool). For transketolase, the increased levels of protein expression in a stool sample may also be shown, such as by using mass spectrometry.

An instance wherein a control patient exhibited high calprotectin expression in stool whereas the expression of two of our novel IBD biomarkers, Annexin A3 and LTA4H, had correctly identified the patient as a control was observed. Annexin A3 and LTA4H may have increased specificity when compared with calprotectin.

It was observed that a portion (23%) of proteins demonstrate opposite relative expression for IBD compared to control between the two proteomic datasets. This can be explained in part by the difference in subcellular localization under assessment in each dataset. For example, complement C3 is increased in IBD within the MLI but decreased in the biopsy dataset, and the tissue macrophages or epithelial cells within the biopsy may be releasing C3 into the extracellular space, hence being detected at relatively higher levels within the MLI of IBD patient samples than in control samples, highlighting the insight gained by the multi-omic comparison. Amongst the 26 proteins, 5 (ELANE, CORO1A, CTSG, ANXA3 and C3) are involved in leukocyte mediated immunity.

The elevated expression of TKT that was observed in IBD patients compared to controls may be a response toward reducing the ROS-induced damage that is observed within the colon of IBD patients³³.

As for catalase, a significant difference in catalase expression between CD and UC at the MLI was not observed (FIG. 12B). However, there is significant elevation of catalase in MLI from both macroscopic (CoA) and microscopic (CoN) inflammatory aspirates from IBD patients when compared with controls. Similar to TKT, the elevated levels of catalase may be to protect cells from the damage caused by hydrogen peroxide which is elevated in the inflamed mucosa of IBD patients³⁶.

Leukotriene A-4 hydrolase (LTA4H) is the only protein included in both the IBD and UC extent biomarker panels. LTA4H catalyzes the biosynthesis of leukotriene B4, which in turn is a potent neutrophilic chemoattractant and has been implicated in chronic inflammation³⁷.

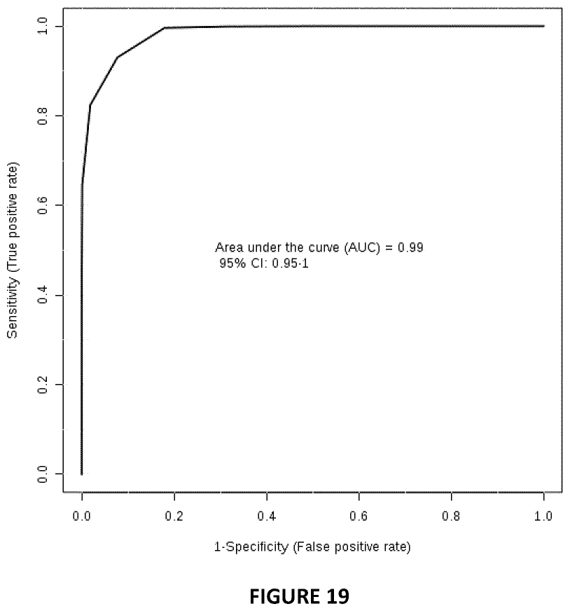

Proteins TXNDC17, TMSB10 and VASP are also identified as biomarkers for IBD diagnosis, as part of the panel for disease extent in UC. Moreover, proteins TXNDC17, TMSB10 and VASP may also be used to indicate the presence of IBD in a patient, due to its different relative expression levels when compared to that of a healthy subject, as shown in FIG. 19. Interestingly, TXNDC17 can regulate TNF-α signaling³⁸; TNF-α is an important signaling molecule in inflammation, and is the target of anti-TNF-α agents that are utilized for the induction and maintenance of remission in CD. Unlike the role of TXNDC17, TMSB10 and VASP are involved in cytoskeleton organization^{39, 40}. Perhaps elevated VASP observed in inflamed UC is associated with altered host-microbe interactions. Despite both having roles in cytoskeletal organization, they demonstrate opposite expression trends in inflamed tissue compared to uninflamed tissue (FIG. 14).

The extent of disease in UC patients can indicate response to therapy, since healing progresses proximally to distally through the colon. Considering that many pediatric UC patients have pancolitis²⁵, a non-invasive biomarker indicating a status of pancolitis vs. non-pancolitis is beneficial as it may permit assessment of response to therapy without the need for colonoscopy.

In summary, this study identified biomarkers that are better at predicting IBD than calprotectin at the MLI. Elevated expression of CAT, Annexin A3 and LTA4H, identified as biomarker candidates at the MLI was reflected in stool samples showing that non-invasively obtained samples may also show similar protein expression levels of the four biomarkers for determination of the presence of IBD. Further, a panel of UC extent of disease biomarkers was identified, classifying 100% of patients with pan/non-pancolitis; this panel was developed by comparison of UC patients with and without macroscopic inflammation, and thus at a level similar in nature to what is observed by endoscopy.

The description of the present invention has been presented for purposes of illustration but is not intended to be exhaustive or limited to the disclosed embodiments. Many modifications and variations will be apparent to those of ordinary skill in the art.

### REFERENCES

1. Vizcaino J A, Csordas A, Del-Toro N, et al. 2016 update of the PRIDE database and its related tools. Nucleic Acids Res 2016; 44:11033.
2. Benchimol E I, Fortinsky K J, Gozdyra P, et al. Epidemiology of pediatric inflammatory bowel disease: a systematic review of international trends. Inflamm Bowel Dis 2011; 17:423-39.
3. Levine A, Koletzko S, Turner D, et al. ESPGHAN revised porto criteria for the diagnosis of inflammatory bowel disease in children and adolescents. J Pediatr Gastroenterol Nutr 2014; 58:795-806.
4. van Rheenen P F, Van de Vijver E, Fidler V. Faecal calprotectin for screening of patients with suspected inflammatory bowel disease: diagnostic meta-analysis. BMJ 2010; 341:c3369.
5. Henderson P, Anderson N H, Wilson D C. The diagnostic accuracy of fecal calprotectin during the investigation of suspected pediatric inflammatory bowel disease: a systematic review and meta-analysis. Am J Gastroenterol 2014; 109:637-45.
6. Mendoza J L, Abreu M T. Biological markers in inflammatory bowel disease: practical consideration for clinicians. Gastroenterol Clin Biol 2009; 33 Suppl 3:S158-73.
7 Merras-Salmio L, Kolho K L, Pelkonen A S, et al. Markers of gut mucosal inflammation and cow's milk specific immunoglobulins in non-IgE cow's milk allergy. Clin Transl Allergy 2014; 4:8.
8. Orivuori L, Mustonen K, de Goffau M C, et al. High level of fecal calprotectin at age 2 months as a marker of intestinal inflammation predicts atopic dermatitis and asthma by age 6. Clin Exp Allergy 2015; 45:928-39.
9. Zhu Q, Li F, Wang J, et al. Fecal Calprotectin in Healthy Children Aged 1-4 Years. PLoS One 2016; 11:e0150725.
10. Danese S, Fiocchi C. Ulcerative colitis. N Engl J Med 2011; 365:1713-25.
11. Stange E F, Travis S P, Vermeire S, et al. European evidence-based Consensus on the diagnosis and management of ulcerative colitis: Definitions and diagnosis. J Crohns Colitis 2008; 2:1-23.
12. Starr A E, Deeke S A, Ning Z, et al. Proteomic analysis of ascending colon biopsies from a paediatric inflammatory bowel disease inception cohort identifies protein biomarkers that differentiate Crohn's disease from UC. Gut 2016.
13. Hyams J, Markowitz J, Otley A, et al. Evaluation of the pediatric crohn disease activity index: a prospective multicenter experience. J Pediatr Gastroenterol Nutr 2005; 41:416-21.
14. Turner D, Otley A R, Mack D, et al. Development, validation, and evaluation of a pediatric ulcerative colitis activity index: a prospective multicenter study. Gastroenterology 2007; 133:423-32.
15. Levine A, Griffiths A, Markowitz J, et al. Pediatric modification of the Montreal classification for inflammatory bowel disease: the Paris classification. Inflamm Bowel Dis 2011; 17:1314-21.
16. Jimenez-Rivera C, Haas D, Boland M, et al. Comparison of two common outpatient preparations for colonoscopy in children and youth. Gastroenterol Res Pract 2009; 2009:518932.
17. Mottawea W, Chiang C-K, Mühlbauer M, et al. Altered intestinal microbiota-host mitochondria crosstalk in new onset Crohn's disease. Nature Communications 2016; 7:13419.
18. Ong S E, Mann M. A practical recipe for stable isotope labeling by amino acids in cell culture (SILAC). Nat Protoc 2006; 1:2650-60.
19. Geiger T, Cox J, Ostasiewicz P, et al. Super-SILAC mix for quantitative proteomics of human tumor tissue. Nat Methods 2010; 7:383-5.
20. Wisniewski J R, Zougman A, Mann M. Combination of FASP and StageTip-based fractionation allows in-depth analysis of the hippocampal membrane proteome. J Proteome Res 2009; 8:5674-8.
21. Cox J, Matic I, Hilger M, et al. A practical guide to the MaxQuant computational platform for SILAC-based quantitative proteomics. Nat Protoc 2009; 4:698-705.
22. Xia J, Sinelnikov I V, Han B, et al. MetaboAnalyst 3.0—making metabolomics more meaningful. Nucleic Acids Res 2015; 43:W251-7.
23. Huang da W, Sherman B T, Lempicki R A. Bioinformatics enrichment tools: paths toward the comprehensive functional analysis of large gene lists. Nucleic Acids Res 2009; 37:1-13.
24. Harris P A, Taylor R, Thielke R, et al. Research electronic data capture (REDCap)—a metadata-driven methodology and workflow process for providing translational research informatics support. J Biomed Inform 2009; 42:377-81.
25. Sauer C G, Kugathasan S. Pediatric inflammatory bowel disease: highlighting pediatric differences in IBD. Gastroenterol Clin North Am 2009; 38:611-28.
26. Vernier-Massouille G, Balde M, Salleron J, et al. Natural history of pediatric Crohn's disease: a population-based cohort study. Gastroenterology 2008; 135:1106-13.
27. Wlodarska M, Kostic A D, Xavier R J. An integrative view of microbiome-host interactions in inflammatory bowel diseases. Cell Host Microbe 2015; 17:577-91.
28. Rifai N, Gillette M A, Carr S A. Protein biomarker discovery and validation: the long and uncertain path to clinical utility. Nat Biotechnol 2006; 24:971-83.
29. Presley L L, Ye J, Li X, et al. Host-microbe relationships in inflammatory bowel disease detected by bacterial and metaproteomic analysis of the mucosal-luminal interface. Inflamm Bowel Dis 2012; 18:409-17.
30. Marshall K W, Mohr S, Khettabi F E, et al. A blood-based biomarker panel for stratifying current risk for colorectal cancer. Int J Cancer 2010; 126:1177-86.
31. Lassen N, Black W J, Estey T, et al. The role of corneal crystallins in the cellular defense mechanisms against oxidative stress. Semin Cell Dev Biol 2008; 19:100-12.
32. Bentz S, Pesch T, Wolfram L, et al. Lack of transketolase-like (TKTL) 1 aggravates murine experimental colitis. Am J Physiol Gastrointest Liver Physiol 2011; 300:G598-607.
33. Cao W, Vrees M D, Kirber M T, et al. Hydrogen peroxide contributes to motor dysfunction in ulcerative colitis. Am J Physiol Gastrointest Liver Physiol 2004; 286:G833-43.
34. Roozendaal C, Zhao M H, Horst G, et al. Catalase and alpha-enolase: two novel granulocyte autoantigens in inflammatory bowel disease (IBD). Clin Exp Immunol 1998; 112:10-6.
35. Sabery N, Bass D. Use of serologic markers as a screening tool in inflammatory bowel disease compared with elevated erythrocyte sedimentation rate and anemia. Pediatrics 2007; 119:E193-E199.
36. Strus M, Gosiewski T, Fyderek K, et al. A role of hydrogen peroxide producing commensal bacteria present in colon of adolescents with inflammatory bowel disease in perpetuation of the inflammatory process. J Physiol Pharmacol 2009; 60 Suppl 6:49-54.
37. Palmer R M, Stepney R J, Higgs G A, et al. Chemokinetic activity of arachidonic and lipoxygenase products on leuocyctes of different species. Prostaglandins 1980; 20:411-8.
38. Jung Y, Kim H, Min S H, et al. Dynein light chain LC8 negatively regulates NF-kappaB through the redox-dependent interaction with IkappaBalpha. J Biol Chem 2008; 283:23863-71.
39. Yu F X, Lin S C, Morrison-Bogorad M, et al. Effects of thymosin beta 4 and thymosin beta 10 on actin structures in living cells. Cell Motil Cytoskeleton 1994; 27:13-25.
40. Ke Y, Tan Y, Wei N, et al. Yersinia protein kinase A phosphorylates vasodilator-stimulated phosphoprotein to modify the host cytoskeleton. Cell Microbiol 2015; 17:473-85.
41. Lee S Y, Gertler F B, Goldberg M B. Vasodilator-stimulated phosphoprotein restricts cell-to-cell spread of Shigella flexneri at the cell periphery. Microbiology 2015; 161:2149-60.
42. Castellano F, Le Clainche C, Patin D, et al. A WASp-VASP complex regulates actin polymerization at the plasma membrane. Embo Journal 2001; 20:5603-5614.

What is claimed is:

1. A method for determining a presence of inflammatory bowel disease in a subject comprising:
providing a gut sample obtained from a subject;
measuring a level in said gut sample of one or more proteins, wherein said one or more proteins comprises at least one of: leukotriene A-4 hydrolase, catalase, transketolase, thioredoxin domain containing protein 17, vasodilator-stimulated phosphoprotein and thymosin beta-10; and
comparing said measured level to a predetermined protein level to provide an indication of presence of disease.

2. The method as defined in claim 1, wherein said one or more proteins comprises leukotriene A-4 hydrolase and wherein a measured level in said gut sample of leukotriene A-4 hydrolase higher than a predetermined protein level of leukotriene A-4 hydrolase corresponding to a healthy subject is indicative of disease.

3. The method as defined in claim 1 or claim 2, wherein said one or more proteins comprises catalase and wherein a measured level in said gut sample of catalase higher than a predetermined protein level of catalase corresponding to a healthy subject is indicative of disease.

4. The method as defined in any one of claims 1 to 3, wherein said one or more proteins comprises transketolase and wherein a measured level in said gut sample of transketolase higher than a predetermined protein level of transketolase corresponding to a healthy subject is indicative of disease.

5. The method as defined in any one of claims 1 to 4, wherein said measuring comprises measuring each of leukotriene A-4 hydrolase, catalase, transketolase and annexin A3.

6. The method as defined in claim 1, wherein said measuring comprises measuring a level in said gut sample of two or more proteins, wherein said two or more proteins comprises at least two of: leukotriene A-4 hydrolase, catalase, transketolase, annexin A3, thioredoxin domain containing protein 17, vasodilator-stimulated phosphoprotein and thymosin beta-10.

7. The method as defined in claim 1, wherein said measuring comprises measuring a level in said gut sample of three or more proteins, wherein said three or more proteins comprises at least three of: leukotriene A-4 hydrolase, catalase, transketolase, annexin A3, thioredoxin domain containing protein 17, vasodilator-stimulated phosphoprotein and thymosin beta-10.

8. The method as defined in claim 1, wherein said measuring comprises measuring a level in the gut sample of four proteins or more, wherein said four or more proteins comprises at least four of: leukotriene A-4 hydrolase, catalase, transketolase, annexin A3, thioredoxin domain containing protein 17, vasodilator-stimulated phosphoprotein and thymosin beta-10.

9. The method as defined in any one of claims 1 to 8, wherein said measuring comprises using an immunoassay.

10. The method as defined in claim 9, wherein said immunoassay is ELISA.

11. The method as defined in any one of claims 1 to 10, wherein said measuring comprises using semi-quantitative immunoblotting.

12. The method as defined in any one of claims 1 to 11, wherein said measuring comprises using mass spectrometry.

13. The method as defined in any one of claims 1 to 12, wherein said gut sample is a mucosal luminal interface sample.

14. The method as defined in any one of claims 1 to 13, wherein said gut sample is a stool sample.

15. The method as defined in any one of claims 1 to 14, wherein said subject is a pediatric subject.

16. A method of treating inflammatory bowel disease in a subject comprising:
determining whether said subject has inflammatory bowel disease according to the method of any one of claims 1 to 15 and administrating to said patient a compound pharmaceutically effective against said inflammatory bowel disease.

17. The method as defined in claim 16, wherein said administering comprises administering a pharmaceutically effective amount of a compound selected from aminosalicylates, immunomodulators, anti-integrins, anti-cytokines, enteral feed programs, corticosteroids, antibiotics, monoclonal antibodies or a combination thereof.

18. The method as defined in any one of claims 1 to 17, wherein said subject was determined to have, at a time prior to obtaining said gut sample, inflammatory bowel disease, and wherein said comparing to provide an indication of presence of disease is to further determine if said disease is in remission or if remission is maintained.

19. A method for determining a presence of pancolitis in a subject with ulcerative colitis comprising:
providing a gut sample obtained from a subject with ulcerative colitis;
measuring in said gut sample one or more proteins, wherein said one or more proteins comprises at least one of: leukotriene A-4 hydrolase, thioredoxin domain containing protein 17, vasodilator-stimulated phosphoprotein, and thymosin beta-10; and
comparing said measured level to a predetermined protein level to provide an indication of the presence or absence of pancolitis.

20. The method as defined in claim 19, wherein at least one of:
said one or more proteins comprises leukotriene A-4 hydrolase and wherein a measured level in said gut sample of leukotriene A-4 hydrolase higher than a predetermined protein level of leukotriene A-4 hydrolase corresponding to a subject without pancolitis is indicative of pancolitis;
said one or more proteins comprises thioredoxin domain containing protein 17 and wherein a measured level in said gut sample of thioredoxin domain containing protein 17 higher than a predetermined protein level of thioredoxin domain containing protein 17 corresponding to a subject without pancolitis is indicative of pancolitis;
said one or more proteins comprises vasodilator-stimulated phosphoprotein and wherein a measured level in said gut sample of vasodilator-stimulated phosphoprotein higher than a predetermined protein level of vasodilator-stimulated phosphoprotein corresponding to a subject without pancolitis is indicative of pancolitis; and
said one or more proteins comprises thymosin beta-10 and wherein a measured level in said gut sample of thymosin beta-10 lower than a predetermined protein level of thymosin beta-10 corresponding to a subject without pancolitis is indicative of pancolitis.

21. The method as defined in claim 19 or claim 20, wherein said one or more proteins comprises leukotriene A-4 hydrolase and wherein a measured level in said gut sample of leukotriene A-4 hydrolase higher than a predetermined protein level of leukotriene A-4 hydrolase corresponding to a subject without pancolitis is indicative of pancolitis.

22. The method as defined in any one of claims 19 to 21, wherein said one or more proteins comprises thioredoxin domain containing protein 17 and wherein a measured level in said gut sample of thioredoxin domain containing protein 17 higher than a predetermined protein level of thioredoxin domain containing protein 17 corresponding to a subject without pancolitis is indicative of pancolitis.

23. The method as defined in any one of claims 19 to 22, wherein said one or more proteins comprises vasodilator-stimulated phosphoprotein and wherein a measured level in said gut sample of vasodilator-stimulated phosphoprotein higher than a predetermined protein level of vasodilator-stimulated phosphoprotein corresponding to a subject without pancolitis is indicative of pancolitis.

24. The method as defined in any one of claims 19 to 23, wherein said one or more proteins comprises thymosin beta-10 and wherein a measured level in said gut sample of thymosin beta-10 lower than a predetermined protein level of thymosin beta-10 corresponding to a subject without pancolitis is indicative of pancolitis.

25. The method as defined in any one of claims 19 to 24, wherein said measuring comprises measuring each of leukotriene A-4 hydrolase, thioredoxin domain containing protein 17, vasodilator-stimulated phosphoprotein, and thymosin beta-10.

26. The method as defined in claim 19, wherein said measuring comprises measuring a level in said gut sample of two or more proteins, wherein said two or more proteins comprises at least two of: leukotriene A-4 hydrolase, thioredoxin domain containing protein 17, vasodilator-stimulated phosphoprotein, and thymosin beta-10.

27. The method as defined in claim 19, wherein measuring comprises measuring a level in said gut sample of three or more proteins, wherein said three or more proteins comprises at least three of: leukotriene A-4 hydrolase, thioredoxin domain containing protein 17, vasodilator-stimulated phosphoprotein, and thymosin beta-10.

28. The method as defined in any one of claims 19 to 27, wherein said gut sample is a mucosal luminal interface sample.

29. The method as defined in any one of claims 19 to 27, wherein said gut sample is a stool sample.

30. The method as defined in any one of claims 19 to 29, wherein said subject is a pediatric subject.

31. The method as defined in any one of claims 19 to 30, wherein said measuring comprises using an immunoassay.

32. The method as defined in claim 31, wherein said immunoassay is ELISA.

33. The method as defined in any one of claims 19 to 32, wherein said measuring comprises using semi-quantitative immunoblotting.

34. The method as defined in any one of claims 19 to 33, wherein said measuring comprises using mass spectrometry.

35. A method of treating ulcerative colitis in a subject comprising:
determining whether said ulcerative colitis subject has pancolitis or does not have pancolitis according to the method of any one of claims 19 to 34, and administrating to said patient a compound pharmaceutically effective against:
pancolitis;
or ulcerative colitis without pancolitis,
said administration tailored in accordance with said determined presence or absence of pancolitis.

36. The method as defined in claim 35, wherein said administering comprises administering a pharmaceutically effective amount of aminosalicylates, immunomodulators, anti-integrins, anti-cytokines, enteral feed programs, corticosteroids, antibiotics, monoclonal antibodies or a combination thereof.

37. A method for determining the efficacy of a treatment of inflammatory bowel disease, said treatment comprising the administration of aminosalicylates, immunomodulators, anti-integrins, anti-cytokines, enteral feed programs, corticosteroids, antibiotics, monoclonal antibodies or a combination thereof, said method comprising:
measuring a level in an indicative gut sample, obtained from a patient, of one or more proteins, wherein said one or more proteins comprises at least one of: leukotriene A-4 hydrolase, catalase, transketolase, thioredoxin domain containing protein 17, vasodilator-stimulated phosphoprotein and thymosin beta-; and
comparing said measured level to at least one of:
a corresponding protein level measured in a reference gut sample taken from said patient at a time prior to when said indicative gut sample was obtained;
a predetermined protein level;
a corresponding protein level associated with responders; and
a corresponding protein level associated with non-responders; and
assessing responsiveness to treatment as a function of said comparison.

38. The method as defined in claim 37, wherein said corresponding protein levels of responders is an average of responders' protein levels of said corresponding protein, and wherein said corresponding protein levels of non-responders is an average of non-responders' protein levels of said corresponding protein.

39. The method as defined in claim 37 or claim 38, wherein said measuring comprises performing an assay.

40. The method as defined in any one of claims 37 to 39, wherein said patient is a pediatric patient.

41. A method for determining the efficacy of a treatment of ulcerative colitis, said treatment comprising the administration of aminosalicylates, immunomodulators, anti-integrins, anti-cytokines, enteral feed programs, corticosteroids, antibiotics, monoclonal antibodies or a combination thereof, said method comprising:
measuring a level, in a gut sample obtained from a patient with ulcerative colitis, of one or more proteins, wherein said one or more proteins comprises at least one of: leukotriene A-4 hydrolase, thioredoxin domain containing protein 17, vasodilator-stimulated phosphoprotein, and thymosin beta-10;
comparing said measured level in said gut sample to a predetermined protein level to indicate the presence or absence of pancolitis; and
assessing responsiveness of treatment with reference to a prior health condition of said patient wherein one of:
said assessment is indicative of responsiveness to treatment when said prior health condition was that said patient had pancolitis and said comparing said measured level in said gut sample indicates an absence of pancolitis; and
said assessment is indicative of non-responsiveness to treatment when said prior health condition was that said patient did not have pancolitis and said comparing said measured level in said gut sample indicates a presence of pancolitis.

42. The method as defined in claim 41, wherein at least one of:
said one or more proteins comprises leukotriene A-4 hydrolase and wherein a measured level in said gut sample of leukotriene A-4 hydrolase higher than a protein level of leukotriene A-4 hydrolase for a subject without pancolitis is indicative of pancolitis;
said one or more proteins comprises thioredoxin domain containing protein 17 and wherein a measured level in said gut sample of thioredoxin domain containing protein 17 higher than a protein level of thioredoxin domain containing protein 17 for a subject without pancolitis is indicative of pancolitis;
said one or more proteins comprises vasodilator-stimulated phosphoprotein and wherein a measured level in said gut sample of vasodilator-stimulated phosphoprotein higher than a protein level of vasodilator-stimulated phosphoprotein for a subject without pancolitis is indicative of pancolitis; and
said one or more proteins comprises thymosin beta-10 and wherein a measured level in said gut sample of thymosin beta-10 lower than a protein level of thymosin beta-10 for a subject without pancolitis is indicative of pancolitis.

43. The method as defined in claim 41 or claim 42, wherein said measuring comprises performing an assay.

44. The method as defined in any one of claims 41 to 43, wherein said patient is a pediatric patient.

45. A method for determining a presence of inflammatory bowel disease in a subject comprising:
providing a gut sample obtained from a subject;
measuring a level in said gut sample of two or more proteins, wherein said two or more proteins comprises at least two of: leukotriene A-4 hydrolase, Annexin A3, catalase, transketolase, thioredoxin domain containing protein 17, vasodilator-stimulated phosphoprotein and thymosin beta-10; and
comparing said measured level to a predetermined protein level to provide an indication of presence of disease.

46. The method as defined in claim 45, wherein said two or more proteins comprises leukotriene A-4 hydrolase and wherein a measured level in said gut sample of leukotriene A-4 hydrolase higher than a predetermined protein level of leukotriene A-4 hydrolase corresponding to a healthy subject is indicative of disease.

47. The method as defined in claim 45 or claim 46, wherein said two or more proteins comprises catalase and wherein a measured level in said gut sample of catalase higher than a predetermined protein level of catalase corresponding to a healthy subject is indicative of disease.

48. The method as defined in any one of claims 45 to 47, wherein said two or more proteins comprises transketolase and wherein a measured level in said gut sample of transketolase higher than a predetermined protein level of transketolase corresponding to a healthy subject is indicative of disease.

49. The method as defined in any one of claims 45 to 48, wherein said measuring comprises measuring each of leukotriene A-4 hydrolase, catalase, transketolase and annexin A3.

50. The method as defined in claim 45, wherein said measuring comprises measuring a level in said gut sample of three or more proteins, wherein said three or more proteins comprises at least three of: leukotriene A-4 hydrolase, catalase, transketolase, annexin A3, thioredoxin domain containing protein 17, vasodilator-stimulated phosphoprotein and thymosin beta-10.

51. The method as defined in claim 45, wherein said measuring comprises measuring a level in the gut sample of four proteins or more, wherein said four or more proteins comprises at least four of: leukotriene A-4 hydrolase, catalase, transketolase, annexin A3, thioredoxin domain containing protein 17, vasodilator-stimulated phosphoprotein and thymosin beta-10.

52. The method as defined in any one of claims 45 to 51, wherein said measuring comprises using an immunoassay.

53. The method as defined in claim 52, wherein said immunoassay is ELISA.

54. The method as defined in any one of claims 45 to 53, wherein said measuring comprises using semi-quantitative immunoblotting.

55. The method as defined in any one of claims 45 to 54, wherein said measuring comprises using mass spectrometry.

56. The method as defined in any one of claims 45 to 55, wherein said gut sample is a mucosal luminal interface sample.

57. The method as defined in any one of claims 45 to 56, wherein said gut sample is a stool sample.

58. The method as defined in any one of claims 45 to 57, wherein said providing an indication of presence of disease further indicates if the disease is in relapse.

59. The method as defined in any one of claims 45 to 57, wherein said providing an indication of presence of disease further indicates if the disease is in remission.
